(19) United States
(12) Patent Application Publication
(10) Pub. No.: US 2014/0255462 A1
(43) Pub. Date: Sep. 11, 2014

(54) Xenotransplant Compositions and Methods of Stroke Therapy

(71) Applicant: Neurotrophincell Pty Ltd., Parkside (AU)

(72) Inventors: Robert Bartlett Elliott, Auckland (NZ); Stephen John Martin Skinner, Auckland (NZ); Alfred Vasconcellos, Cranston, RI (US); Dwaine Emerich, Glocester, RI (US); Cesar Venturina Borlongan, Augusta, GA (US); Christopher Edward Williams, Auckland (NZ)

(73) Assignee: Neurotrophincell Pty Ltd., Parkside (AU)

(21) Appl. No.: 14/049,030

(22) Filed: Oct. 8, 2013

Related U.S. Application Data
(63) Continuation of application No. 12/858,180, filed on Aug. 17, 2010, now abandoned.
Application No. 12/858,180 is a continuation of application No. 12/248,410, filed on Oct. 9, 2008, now Pat. No. 7,984,873.
Application No. 12/248,410 is a continuation-in-part of application No. 11/036,202, filed on Jan. 14, 2005, now abandoned.
Application No. 11/036,202 is a continuation-in-part of application No. 09/959,560, filed on Oct. 30, 2001, now abandoned.

(51) Int. Cl. A61K 35/30; A61K 9/48; A61K 9/00

(52) U.S. Cl. CPC A61K 35/30; A61K 9/0085; A61K 9/4816
USPC 424/424; 424/423; 424/93.7; 424/93.21

(57) ABSTRACT

Disclosed are implant compositions and methods for treatment of neurological diseases of the central nervous system of a mammal.

### CROSS-REFERENCE TO RELATED APPLICATIONS

The present application is a continuation of U.S. patent application Ser. No. 12/858,180, filed Aug. 17, 2010 (pending; Atty. Dkt. No. 36697.29); which was a continuation of U.S. patent application Ser. No. 12/248,410, filed Oct. 9, 2008 (abandoned; Atty. Dkt. No. 36697.26); which was a continuation-in-part of U.S. patent application Ser. No. 11/036,202, filed Jan. 14, 2005 (abandoned; Atty. Dkt. No. 36697.5); which was a continuation-in-part of U.S. patent application Ser. No. 10/757,428, filed Jan. 15, 2004 (abandoned; Atty. Dkt. No. 36697.3); which was a continuation-in-part of U.S. patent application Ser. No. 09/959,560, filed Oct. 30, 2001 (abandoned; Atty. Dkt. No. 36697.4); which was a 35 U.S.C. §371 nationalization of PCT International Patent Application No. PCT/NZ00/00064, filed Apr. 28, 2000 (abandoned); which claims priority to New Zealand Patent Application No. NZ 335553L, filed Apr. 30, 1999 (abandoned). The entire content of each of which is specifically incorporated herein in its entirety by express reference thereto.

### FIELD OF THE INVENTION

This invention relates to a composition for treatment of some neurological diseases of the central nervous system of a mammal and, more particularly, to compositions including living cells derived from a mammal, and in particular to a composition and method of use employing the living cells to express factors, over a period, capable of having a desired effect on the central nervous system.

### BACKGROUND OF THE INVENTION

Significant neurodegenerative diseases of the central nervous system (CNS) include Alzheimer's disease (AZ), multiple sclerosis (MS) and Parkinson's disease (PD). In the United States and Europe alone, the incidence of AZ is estimated at 8 million; MS at 0.7 million, and PD at 1.5 million. There are of course many other diseases; epilepsy, Huntington's chorea, stroke, and so on. At this time, all available treatments would appear to be palliative rather than restorative and the inevitable progress of these diseases is slowed, perhaps, but not reversed.

The assumption that neurones cannot regenerate has constrained past approaches for treatments of diseases of the central nervous system. Furthermore, therapy of the central nervous system (CNS) is more difficult than for the remainder of the body in part because of the “blood-brain barrier”—which is a concept used to describe a functional obstacle to the entry of some materials including therapeutic materials from the systemic circulation. The barrier resides, in functional terms, around all (normal) capillary structures within the CNS. Morphologically, altered pinocytotic behaviour and tight junctions of the endothelial cells are characteristic. Introduction of foreign substances directly into the CNS, such as into the ventricles is a good deal more difficult, unpleasant, and dangerous than taking a pill four times daily. In addition, a particular and quite separate version of “lymph circulation” within the CNS—the circulation of cerebrospinal fluid—tends to remove any material that does cross the barrier. Lymphatics themselves do not extend to the CNS.

There is also a knowledge barrier. For example a widely-held belief is that the cerebrospinal fluid does not perfuse the substance of the brain in a manner capable of carrying materials about, while a few, including ourselves, believe that it does provide an effective perfusion medium capable of distributing trophic factors about most, if not all of the parenchyma of the CNS making use of white matter tracts, perivascular spaces and the like. There has been experimental evidence for that widely held belief, e.g., Brightman & Reese, or Blasberg, Patlack & Fenstermacher, (cited in WM Pardridge, “Transnasal and intraventricular delivery of drugs” in “Peptide drug delivery to the brain” ed. WM Pardridge, New York: Raven Press, 1991) involving limited distances achieved by the intraventricular infusion of a selection of traceable compounds. Most prior art known to us appear to be based on local diffusion, such as U.S. Pat. No. 5,853,385, or U.S. Pat. No. 5,573,528 “Implanting devices for the focal release of neuroinhibitory compounds” to Aebischer & Tresco. Krewson et al. (Brain Res., 1995 May 22 680(1-2):196-206 state that nerve growth factor travelled only 2-3 mm from a polymer insert through rat brain tissue. This paper also exemplifies the “single-factor” approach. See later. A knowledge gap also extends in relation to the interplay between trophic substances (such as insulin growth factors including IGF-II and the like, also nerve growth factor or NGF,) and their normal regulation and site or sites of production at different stages of life including the fetus. Walter H J et al., (Endocrinology, 140(1) 520-32) considers that IGF-II secretion from the choroid plexus of an injured rat brain is raised as a response to injury, “resulting in an increased transport of the peptide to the wound”.

An increasing number of conditions of the central nervous system capable of responding to therapy are being recognized. It is interesting to note that cerebrospinal fluid production is impaired in a number of such conditions and furthermore it is possible that there is a loss of paracrine factors such as growth factors, in the case of specific diseases.

In many cases the indicated therapeutic agent for a restoration therapy or the like is a naturally occurring cell secretion, for example a polypeptide (such as IGFII) rather than an exogenous substance such as an antibiotic derived from a fungus or bacterium. Substantially continuous application throughout the entire CNS over a long period is acceptable in most of these treatments.

Within the patent literature, Patrick Aebischer and associates have filed many patents dealing with implants, including both live cells and manufactured slow-release formulations into specified parts of the CNS; for example U.S. Pat. No. 5,389,535, for manufacturing a tubular cell-carrying implant. WO 99/56770 to Chang is possibly most similar to the present application, in that Chang teaches the injection of microcapsules holding specified live cells, capable of releasing an enzyme lacking in a lysosomal storage disease, into a ventricle. Cells known as “neural stem cells” are used by Carpenter in U.S. Pat. No. 5,968,829 to CytoTherapeutics, Inc.; such cells are undifferentiated cells capable of evolving into either neurones or glial cells. A commercial application was absent. Many documents (e.g., U.S. Pat. No. 5,898,066 for trophic factors (axogenesis factors), WO 99/36565 (human ependymin), U.S. Pat. No. 5,573,528 (neuroinhibitory compounds such as GABA for control of involuntary movement) deal with specific substances. Gage et al., (U.S. Pat. No. 5,762,926) exemplifies genetically modified live-cell grafts, and Holland et al., (U.S. Pat. No. 5,550,050) describes exposure of live cells to restrictive conditions prior to implantation; both so that the resulting implant functions in the intended manner.

There is little published material dealing with “factors leading to rejuvenation” and no patents take advantage of the differentiated, very active cells of the choroid plexus.

The problem to be solved is to identify an effective treatment for at least one neurological disease.

### DEFINITIONS

A “neurological disease” covers any disorder of the central nervous system. It may for example be a global neurodegenerative disease, such as ageing, vascular disease, Alzheimer's disease, or the more localised Parkinson's disease, or the autoimmune disease multiple sclerosis (MS), it may be a result of an injury, such as a stroke, anoxia/asphyxia, or physical injury such as from a blow to the head, it may be a result of exposure to local (e.g., meningitis) or systemic toxins, and it may be neoplastic. It may be genetically based, such as Huntington's chorea, or a disorder of metabolism such as lysosomal storage disease.

There is a group of “global neurodegenerative diseases” including AZ and others, affecting the elderly, the usual pattern of response to acute injury (such as ischaemia), affecting any age group including stroke victims and car accident victims, autoimmune diseases such as MS, PD, and certain diseases, including deficiencies of metabolism, of neonates and fetuses. Indeed PD may be more global than is currently appreciated. The known defects in and around the basal ganglia may be reflected elsewhere.

By “restorative effect” we include any beneficial modification of the disease process, including palliative, restorative, or proliferative effects acting on neural tissue, glia, or vascular elements. We tend to use “trophic” and “growth” factors interchangeably.

By “rejuvenation” we mean attempts to reverse changes in a brain commonly considered to be the usual, if not the normal consequences of ageing, such as loss of volume, loss or atrophy of neurones, loss of memory, and loss of ability to cope with complex sensory inputs. Rejuvenation could also comprise restorative effects on existing neurones, neural rescue as required after an asphyxic episode, or “sick neurons.”

All references, including any patents or patent applications, cited in this specification are hereby incorporated into the specification by reference (without any admission being made that any reference constitutes prior art against the present U.S. application).

### OBJECT OF THE INVENTION

It is an object of this invention to provide apparatus and/or material, and/or means for CNS therapy based on xenotransplantation of choroid plexus epithelium, or at least to provide the public with a useful choice.

### BRIEF SUMMARY OF INVENTION

In a first broad aspect this invention provides a pharmaceutical composition, comprising an implant for implantation into the brain of a recipient mammal suffering from a neurological disease, wherein the implant comprises living cells, derived from epithelial cells of the choroid plexus of another mammal, and the living cells are capable of expressing at least one product having a beneficial effect on the neurological disease into the brain of the recipient mammal.

Preferably, all the living cells are derived from epithelial cells of the choroid plexus; alternatively some of the cells may be derived from other tissues of the choroid plexus or from other sources.

Preferably, the pharmaceutical composition is modified so as to be capable of survival following its introduction within the brain of the mammal while producing the therapeutic agent, so that treatment over an extended period can be applied.

Preferably, the living cells are encapsulated within a biocompatible capsule, the wall of which is at least partially composed of a semi-permeable membrane capable of admitting metabolites for sustaining the cells, capable of blocking access by factors of the immune system of the recipient mammal, and capable of allowing an effective amount of one or more expressed products to exit from the implant.

Preferably, the ingress of any substances capable of controlling the rate of release of the therapeutic agent is also permitted.

Preferably, the biocompatible capsule has an inner layer comprised substantially of a laminin or the like; the laminin serving as a physical substrate for the at least one living cell thereby providing orientation and support for the at least one cell.

Preferably, the biocompatible capsule comprises a globular containment means capable of holding at least one cell.

More preferably, the biocompatible capsule comprises an extended tubular containment means capable of holding at least one cell; the implant being capable of placement within a ventricle of the brain of the recipient mammal, so that the substance of the brain receives an effective amount of at least one product carried by means of a flow of cerebrospinal fluid.

One preferred physical substrate is shaped like a hollow dialysis tube which is capable in use of holding living tissue (as previously described in this section) within a space within the CNS.

Another possible physical substrate comprises a closed meshed structure capable of retaining cell groups inside biocompatible capsules within the closed structure so that the entire structure may be removed as a unit.

Preferably, at least one living epithelial cell is taken from the choroid plexus of a fetal or neonatal mammal having a selected age, so that the at least one living cell has a predicted capability for expressing at least one product capable of having a beneficial effect on a neurological disease, so that the recipient mammal may experience a beneficial effect.

Preferably, the donor mammal is a non-human mammal.

Conveniently, the donor mammal or at least the living material is free of infectious agents and preferably the donor mammal is from a stock kept under germ-free conditions.

Optionally, the at least one living cell has undergone subsequent modification in order to increase the production of at least one product capable of having a beneficial effect on a neurological disease, so that the recipient mammal may experience a beneficial effect.

Optionally, the living material may comprise cultured cells; that is, separated by one or more generations from an initial isolate.

Preferably, treatments such as bFGF may be used to selectively enhance growth in culture.

Therapeutic agents include, but are not limited to, the naturally occurring peptides IGF-II, VEGF, TGF-alpha, NT-3 and bFGF.

Alternatively, the living material comprises cells having a modified complement of genetic material capable of secreting novel peptides.

Alternatively, the factors secreted may include naturally occurring peptides (such as one or more of those previously listed in this section), in altered amounts.

Alternatively, the peptides secreted may include compounds normally secreted elsewhere, such as thyroxine, insulin, or analogues thereof.

Alternatively, the peptides secreted includes sets of peptides secreted during different stages of development, such as peptides characteristic of fetal or neonatal choroid plexus cells, or analogues thereof.

Preferably, the living material is also capable of being controlled by one or more endogenous control agents, or by one or more exogenous control agents.

Preferably, the invention provides a container for transport and distribution, capable of holding at least one implant as previously described in this section, wherein the container also holds a liquid media capable of maintaining the at least one implant in a living condition for a time during transport and storage.

Optionally, the living material is provided in a state of suspended animation, suitable for storage and transport. Preferably, this state is a cryopreserved state, although other forms of providing for the continuation of cell metabolism are included.

A preferred method for implanting at least one implant as previously described in this section within a ventricle includes the steps of selecting a recipient mammal according to need, surgically accessing a lateral ventricle, placing at least a portion of the implant within the ventricle, and optionally removing the implant after a period of treatment.

In a second broad aspect this invention provides a kit of materials for surgical implantation of an implant, as previously described in this section, in the central nervous system of a recipient mammal, the kit of materials includes means for providing a sterile site, means for obtaining surgical access through the cranium to the central nervous system, means for haemostasis, means for placing at least one implant in an intended position, a container holding implants as previously described in this section, means for closing off the surgical access site, and means for dressing the surgical access site, so that a risk of introducing a slow virus infection during the operative procedure is minimized.

Optionally, the kit of materials is restricted to means for placing at least one implant in an intended position and a container holding implants as previously described in this section.

Preferably, the implant is surgically implanted into a ventricle of the central nervous system and preferably into a lateral ventricle by a frontal route so that the cell products expressed from the implant may flow rapidly into at least some regions of the central nervous system.

Optionally, the implant is implanted into a localized area of the central nervous system; the localized area being known to be liable to benefit, in terms of the neurological disease, from the least one product expressed from the implant.

Preferably the implant is capable of removal after the duration of a treatment procedure has expired, or at least once the efficacy of the pharmaceutical composition has become inappropriate.

In a third broad aspect this invention provides a vascularized device or artificial choroid plexus capable of implantation within the body of a mammal to be treated, wherein the vascularized device includes (a) means to connect a first, blood-bearing compartment of the device between an artery and a vein, (b) means to pass a fluid carrying means leading from a second, transudate-bearing compartment of the device to an implantable second end of the fluid carrying means, capable of being implanted into a space within the brain containing cerebrospinal fluid, and (c) internal support means, comprising a permeable wall between the first compartment and the second compartment, capable of supporting at least one living cell of the invention, so that said at least one living cell is bathed in transudate passing from the first compartment to the second compartment and so that said living cell may express trophic factors into the transudate carried into the brain.

In a fourth broad aspect this invention provides a method for causing at least partial rejuvenation of a brain of a mammal by means of xenotransplantation as previously described in this section, wherein the method employs implantation of an implant of choroid plexus cells derived from a fetal or neonatal mammal into the brain.

In a fifth broad aspect the invention provides a method for treating injuries to the central nervous system; the method including the step of inserting a pharmaceutical composition including living tissue (as described previously in this section) into a CSF-filled space within the central nervous system.

### BRIEF DESCRIPTION OF THE DRAWINGS

The following drawings form part of the present specification and are included to further demonstrate certain aspects of the present invention. The invention may be better understood by reference to one or more of these drawings in combination with the text herein.

FIG. 1 shows a diagram of an encapsulated choroid plexus cell preparation, (Example 1);

FIG. 2 is a graph depicting the effect of conditioned media from cultured choroid plexus on neuronal cell viability, wherein *=P<0.0001 versus 0%;**=P<0.0001 versus 0%, 1% and 3%;

FIG. 3 is a graph depicting the effect of conditioned media from cultured choroid plexus on the number of neurite processes in neuronal cell cultured in media supplemented with fetal bovine serum (Control), unsupplemented media (No Supplement), and in media supplemented with choroid plexus conditioned media (CP-CM Supplement);

FIG. 4 is a graph depicting the effect of conditioned media from cultured choroid plexus on the length of outgrowth of neurite processes in neuronal cell cultured in media supplemented with fetal bovine serum (Control), unsupplemented media (No Supplement), and in media supplemented with choroid plexus conditioned media (CP-CM Supplement);

FIG. 5 shows a graph of uptake of dopamine by cells exposed to media previously surrounding a choroid plexus cell preparation;

FIG. 6 is a photomicrograph of an encapsulated choroid plexus cell preparation;

FIG. 7 is a photomicrograph of an encapsulated choroid plexus cell preparation;

FIG. 8 shows a diagram of an example dialysis tube implant for a choroid plexus cell preparation;

FIG. 9 is a graph depicting stroke-induced motor deficits in stroke-only control animals (◯), stroke animals administered control transplant (▪), and stroke animals administered choroid plexus transplants (▴), wherein *=p<0.01;

FIG. 10 is a graph depicting the neurologic impairment as assessed by the Bederson Test observed in stroke-only control animals (▪), stroke animals administered control transplant (□), and stroke animals administered choroid plexus transplants (▪), wherein *=P<0.0001;

FIG. 11 is a graph depicting the mean striatal infarct volume observed in stroke-only control animals (▪), stroke animals administered control transplant (□), and stroke animals administered choroid plexus transplants (▪), wherein *=P<0.05;

FIG. 12 is a graph depicting the effect of a QA lesion (Huntington's model) on limb performance ▪ (control) versus CP treated animals □*P<0.0001;

FIG. 13A, FIG. 13B, FIG. 13C, FIG. 13D, FIG. 13E, FIG. 13F, FIG. 13G, FIG. 13H, FIG. 13I, FIG. 13J, FIG. 13K, FIG. 13L, FIG. 13M, FIG. 13N, FIG. 13O, FIG. 13P, and FIG. 13Q show photomicrographs of brains from QA-lesioned animals that either received empty capsules (FIG. 13A, FIG. 13C, FIG. 13E, FIG. 13G, FIG. 13I, and FIG. 13K) or encapsulated choroid plexus cells (FIG. 13B, FIG. 13D, FIG. 13F, FIG. 13H, FIG. 13J, and FIG. 13L). Encapsulated choroid plexus cells are seen within the grafted striatum (FIG. 13M). These transplanted capsules, when subsequently retrieved and processed for propidium iodide staining, reveal high (>90%) viability of choroid plexus cells (FIG. 13N). Quantitative assessments of lesion volume (FIG. 13O), ChAT (FIG. 13P) and NADPH diaphorase (FIG. 13Q) immunostaining are also presented. Solid bar represents control, while light shade corresponds to lesioned animals that received encapsulated CP cells. *Statistical significance at p<0.05. Bar=500 μm (panels a, b), 100 μm (panels c, d, g, h, k, l, n), and 250 μm (panels e, f, i, j, m);

FIG. 14 is a graph depicting the behavior recovery of rats having a 6-OHDA lesion (Parkinson's model) in CP treated animals ( [symbol] ) versus control ( [symbol] ), *p<0.02; and

FIG. 15 is a graph depicting the increase in tyrosine hydroxylose in rats having a 6-OHDA lesion (Parkinson's model) in CP treated animals ( [symbol] versus control ( [symbol] ), *p<0.05.

### BRIEF DESCRIPTION OF ILLUSTRATIVE EMBODIMENTS

The description of the invention to be provided herein is given purely by way of example and should not to be taken in any way as limiting the scope or extent of the invention.

In summary, the invention (in preferred embodiments and as embodied in the examples) particularly comprises the use of living choroid plexus secretory cells used as an xenobiotic transplant or “artificial choroids plexus” placed most conveniently though not exclusively within the cerebral ventricles. The choroid plexus, situated within each lateral ventricle and also in the roof of the fourth ventricle is described as a highly vascularized substrate covered by epithelial cells which are in contact with the cerebrospinal fluid. A large number of villous processes provide an estimated surface area not including consideration of the apical microvilli of the epithelial (sometimes called ependymal) cells of over 200 square cm (adult human).

The choroid plexus is well-innervated vascular tissue (more correctly an organ) covered with a basement membrane comprising the usual variants of collagen, one or more types of laminin, proteoglycans and other extracellular matrix molecules, which is in turn covered by an unicellular epithelium-like layer and occurring in several consistent sites within the cerebral ventricles. It appears to act as the source of most of the cerebrospinal fluid. Electron microscopy shows that the epithelial cells include a number of specialisations for protein synthesis and export including a dense layer of microvilli adjacent to the ventricle and adjacent to rough endoplasmic reticulum with ribosomes, yet relatively little Golgi apparatus, consistent with polypeptide secretion. Mitochondria are frequent. Underlying the epithelium there are fenestrated endothelial cells of an almost continuous layer of capillaries.

The account of which we approve concerning the circulation of cerebrospinal fluid is as follows: The choroid plexus comprises a well-folded sheet of epithelial cells supplied with an extensive capillary bed, together with “a well-developed adrenergic and cholinergic nerve supply” (Lindvall M et al., Acta Physiologica Scand. Suppl., 1977; 452:77-86). Most CSF originates within the choroid plexus tissue as combined ultrafiltrate and secretion, while a small amount originates in subarachnoid and perivascular spaces. This circulates unidirectionally through the cerebral aqueduct or aqueduct of Sylvius into the fourth ventricle, then through the median foramen of Magendie or lateral apertures of Luschka, then into and around the brain and spinal cord. The fate of most CSF is reabsorption into the blood at the arachnoid villi and through capillary walls. In the adult human about 430-450 mL of CSF is produced daily. Given that about 125-150 mL of fluid is present at any one time it follows that this amount is turned over every 6 or 7 hours. This unidirectional flow model does not include a clear path for putative substances from within neural tissue to reach and have any autoregulatory effect on the choroid plexus lying substantially at the “headwaters”; perhaps these travel via the blood or perhaps there is some reusage of CSF. Carriage of CSF through the parenchyma of the brain includes perivascular spaces, white matter tracts, and the like. Recently, Segal MB reviewed the choroid plexus (in Cell Mol. Neurobiol., 20(2):183-196, 2000) and stated that “the CSF may act as a third circulation conveying substances secreted into the CSF rapidly to many brain regions.” Note the term “rapidly.”

Recent research suggests that the choroid plexus is likely to produce a number of trophic factors that co-ordinate cerebral development and thus anabolic processes. For example Zhang et al., (Neuroscience, 116:373-382, 2003) note that the thyroxin transport protein “transthyretin” (TTR) occurs in choroidal epithelial cells (and may serve as a diagnostic or assay feature to indicate activity of such cells). Age dependence of the trophic factors being produced is quite likely. Our previous research on xenobiotic transplants of pig pancreatic islet cells as an “artificial endocrine pancreas” provided a source of systemically available insulin which is responsive to autoregulation and the present invention provides an analogous approach to treatment of tissues usually regarded as behind the blood-brain barrier and therefore difficult to reach for treatment.

Indeed, Alzheimer's disease is sometimes called “diabetes of the brain”. Whether or not this particular description is accurate, we expect to identify a number of syndromes where an artificial choroid plexus provides a useful form of treatment particularly in that it avoids repeatedly invading the CSF for the purposes of treatment. Myelinisation of axons in the central nervous system, such as during early postnatal life, presumably depends at least in part on atrophic factor.

It would appear that the transplantation of choroid plexus cells behind a mutually protective barrier (e.g., alginate encapsulation) within or near a ventricle would provide a route for the introduction of substances into the CSF without having to cross a blood-brain barrier. Initially we have explored those substances naturally produced by choroid plexus epithelial cells. Known factors include: insulin-like growth factor (IGF-II), transforming growth factor-alpha (TGF-α), retinoic acid (RA) which may be an essential trigger for neural differentiation, perhaps nerve growth factors (NGF), and possibly, because these factors are present in the CSF, vaso-endothelial growth factor (VEGF), and fibroblast growth factor (FGF). The choroid plexus also synthesizes a variety of binding proteins, which act as directed carriers of trophic factors.

A wide variety of conditions may be treated using the choroids plexus transplantable material of the invention. These include conditions in which cells of the recipient's nervous system would benefit by direct or indirect exposure to the secretion of the choroid plexus cells including global neurodegenerative diseases such as Alzheimer's disease (AZ), aging, vascular disease, motor neuron disease (ALS), or the more local disease of Parkinson's disease (PD); autoimmune disorders such as multiple sclerosis (MS), epilepsy, Huntington's disease (HD), inborn errors of metabolism such as Menkes Kinky Hair Syndrome, Wilsons Disease and other neurological disease or disorders.

Alzheimer's disease (AZ) is a complex multi-genic neurodegenerative disorder characterized by progressive impairments in memory, behaviour, language, and visuo-spatial skills, ending ultimately in death. Hallmark pathologies within vulnerable regions include extracellular .beta.-amyloid deposits, intracellular neurofibrallary tangles, synaptic loss, and extensive neuronal cell death. Although many models of the disease have been proposed, no single model of AZ satisfactorily accounts for all neuropathologic findings as well as the requirement of aging for disease onset. The mechanisms of disease progression are equally unclear.

Considerable human genetic evidence has implicated alterations in production or processing of the human amyloid precursor protein (APP) in the etiology of the disease. However, intensive research has indicated that AZ is a multifactorial disease with many different, perhaps overlapping, etiologies.

Thus far, the therapeutic strategies attempted have targeted neurotransmitter replacement or the preservation of normal brain structures, which potentially provide short-time relief but do not prevent neuronal degeneration and death. Thus, there is a need for therapies that prevent neuronal degeneration and death associated with Alzheimer's disease, and provide long-term relief.

Motor neuron disease, or amyotrophic lateral sclerosis (ALS), is a lethal degenerative disorder of motor neurones involving neurone loss in the cortex, brainstem and spinal cord, resulting in progressive paralysis. It occurs in adult life, and the rate of progression of the disease is variable between individuals but linear within an individual. Death within a year or two of onset is the most common course. The expected general population rate is 2/100,000. It is rarely familial, and when it is, it reportedly usually involves mutations on chromosome 21 involving Cu/Zn super oxide dismutase. Other putative genetic lesions have been proposed in a syndrome affecting Ashkenazi Jews involving ALS, schizophrenia and certain blood disorders. These lesions reportedly involve a choroid plexus transport protein, transthyretin on chromosome 18, transforming growth factor beta 3 and others. Usually the disease is sporadic with no known genetic accompaniments.

The relentless progression of the disease has not been halted by any therapy tried so far in man. Neurotrophins are promising candidates to slow the progression of ALS, since they support neuronal survival and regrowth processes. Motor neurones respond to many members of the neurotrophin family and have receptors for them. Work with cell cultures and animal models provide solid support for the hypothesis that neurotrophins can prevent ALS-like neuronal death. However, intrathecal Brain Derived Neurotrophic Factor (BDNF) has already been trialled with disappointing results and unacceptable side effects.

Parkinson's disease is a disorder of the brain characterized by shaking and difficulty with walking, movement, and coordination. The disease is associated with progressive deterioration of the nerve cells of the part of the brain that controls muscle movement (the basal ganglia and the extra pyramidal area). The neurotransmitter dopamine is normally produced in this area, and deterioration of this area of the brain reduces the amount of dopamine available to the body. Insufficient dopamine disturbs the balance between dopamine and other transmitters, such as acetylcholine. Without dopamine, effective neurotransmission is decreased resulting in the loss of muscle function. The exact reason that the cells of the brain deteriorate is unknown.

The disorder may affect one or both sides of the body, with varying degrees of loss of function. Depression also accompanies this disease due to the person's slow loss of muscle function. Symptoms include muscle rigidity, loss of balance, shuffling walk, slow movements, difficulty beginning to walk, freezing of movement, muscle aches, shaking and tremors, changes in facial expression, voice/speech changes, and loss of fine motor skills, frequent falls, and decline in intellectual function.

Parkinson's disease affects approximately 2 out 1000 people, and most often develops after age 50. It occasionally occurs in younger adults and rarely in children. It affects both men and women and is one of the most common neurologic disorders of the elderly. In some cases, the disease occurs within families, especially when it affects young people. Most late onset cases are sporadic. The term “parkinsonism” refers to any condition that involves a combination of the types of changes in movement seen in Parkinson's disease, which happens to be the most common condition causing this group of symptoms. Parkinsonism may be caused by other disorders or by external factors.

There is no known cure for Parkinson's disease. Treatment is aimed at controlling the symptoms. Medications control symptoms primarily by controlling the imbalance of transmitters. Many of the current medications require monitoring due to severe side effects. Deprenyl may provide some improvements to mildly affected patients. Amantadine and/or anticholinergic medications may be used to reduce early or mild tremors. Levodopa is a medication that the body converts to dopamine. It may be used to increase the body's supply of dopamine, which may improve balance and movement. Carbidopa is a medication that reduces the side effects of Levodopa and makes Levodopa work well. Additional medications that reduce symptoms and control side effects of primary treatment medications include antihistamines, antidepressants, dopamine agonists, monoamine oxidase inhibitors, and others. One alternative treatment in the experimental stage is allotransplantation (Fahn et al., Neurology, 52[Suppl 2]:A405; Kopyov et al., Exp. Neurol., 149:97-108 (1998)) and xenotransplantation (Weiss, R. A., Science, 285:1221-1222 (1999)) of embryonic neural tissue into the disease CNS. Problems with this alternative treatment include graft rejection, infection including zoonotic infection, and the ethical issues of using suitable donor tissues.

Therefore, due the present state of treatment of Parkinson's wherein the medications either entail many side effects or the use of grafts has thus far been problematic, there is a need in the art for more effective treatment for Parkinson's disease.

Multiple sclerosis (MS) is a slowly progressing demyelinating disease of the central nervous system which is insidious and characterized by multiple and varied neurological symptoms which exhibit remissions and exacerbations. These repeated episodes of inflammation of the nervous tissue generally occur in the area of the central nervous system, including the brain and spinal cord. The location of the inflammation varies from person to person and from episode to episode. The inflammation destroys the myelin covering of the nerve cells in that area, leaving multiple areas of scar tissue (sclerosis) along the covering of the nerve cells. This results in slowing or blocking the transmission of nerve impulses in afflicted nerves, leading to the symptoms of multiple sclerosis.

Symptoms vary because the location and extent of each attack varies. There is usually a stepwise progression of the disorder, with episodes that last days, weeks, or months alternating with times of reduced or no symptoms (remission). The onset of the disease usually occurs between 20 and 50 years of age with a peak occurring in people 30 years old. MS is believed to be immunological in nature but treatment with immuno-suppressive agents is not advised.

Symptoms of multiple sclerosis include, but are not limited to, weakness of one or more extremities, paralysis of one or more extremities, tremors of one or more extremities, muscle spasticity, muscle atrophy, dysfunctional movement beginning in the legs, numbness, tingling, facial pain, loss of vision, double vision, eye discomfort, rapid eye movements, decreased coordination, loss of balance, dizziness, vertigo, urinary hesitancy, strong urge to urinate, frequent need to urinate, decreased memory, decreased spontaneity, decreased judgment, loss of ability to think abstractly, depression, decreased attention span, slurred speech, and fatigue. Symptoms vary with each attack. They may last days to months, then reduce or disappear, then reoccur periodically.

The prevalence of MS varies widely with location with the highest prevalence reported at higher latitudes in northern Europe and northern North America. The geographic variation has led some to suggest that MS may in part be caused by the action of some environmental factor that is more common in high latitudes.

There is no known cure for multiple sclerosis. There are, however, promising therapies that may decrease exacerbations and delay progression of the disease. Treatment is aimed at controlling symptoms and maintaining function to give the maximum quality of life. Patients with a relapsing-remitting course may be placed on immune modulating therapy that requires injection under the skin or in the muscle once or several times a week. This treatment is in the form of interferon (such as Avonex or Betaseron) or another medicine called glatiramer acetate (Copaxone). Other than protective therapies, steroids are given to decrease the severity of an attack if it occurs. Other medicines include Baclofen, Tizanidine, or Diazepammay may be used to reduce muscle spasticity. Cholinergic medications may be helpful to reduce urinary problems. Antidepressant medications may be helpful for mood or behaviour symptoms. Amantadine may be given for fatigue. There is a need, however, in the art for more effective treatment for multiple sclerosis.

Huntington's disease is an inherited condition characterized by abnormal body movements, dementia, and psychiatric problems. This progressive disease involves wasting (degeneration) of nerve cells in the brain. Huntington's disease is inherited as a single gene lesion on chromosome 4. A trinucleotide CAG repeat region present in the gene that is repeated in multiple copies. There is a significant inverse correlation between the age at which an individual's symptoms of Huntington's disease first become apparent, and the number of CAG repeats in the mutant gene. In other words, the more CAG repeats, often the earlier the age that symptoms first appear. Individuals with childhood (<10 years of age) and juvenile (11-20 years of age) onset of the symptoms of Huntington's disease were found to have the larger CAG trinucleotide repeat lengths in their mutant Huntington's disease gene. The disease may occur earlier and more severely in each succeeding affected generation because the number of repeats can increase. However, the CAG repeat size is not a clinically useful predictor of either the age of onset or the rate of progression in individual patients.

There is no cure for Huntington's disease and there is no known way to stop progression of the disorder. Currently, treatment is aimed at slowing progression and maximizing ability to function for as long as possible. Medications vary depending on the symptoms. Dopamine blockers such as haloperidol or phenothiazine medications may reduce abnormal behaviours and movements. Reserpine and other medications have been used, with varying successes reported. Drugs like Tetrabenazine and Amantidine are used to try to control extra movements. There has been some evidence to suggest that Co-Enzyme Q10 may minimally decrease progression of the disease. Alternative therapies, such as those reliant on antibodies, including antibody-nerve growth factor fusions, have been proposed, yet these therapies are primarily directed to the prevention of further neuronal degeneration. Therefore, there is a need in the art for alternative therapies that provide effective treatment of Huntington's disease.

The conditions to be treated also include injury to the nervous system, particularly the brain, such as pressure resulting in head injury, stroke, anoxia/asphyxia, and injury resulting from CO₂ or CO poisoning.

This invention may also be said broadly comprise the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention is embodied in the foregoing and also envisages constrictions of which the following gives examples only. The following examples are therefore included to demonstrate certain preferred embodiments of the invention. It will be appreciated by those of ordinary skill in the art that the techniques disclosed in the examples that follow, represent techniques discovered by the inventors to function well in the practice of the invention, and thus can be considered to constitute certain preferred modes for its practice. However, those of skill in the art, in light of the present disclosure, will appreciate that changes can be made in the specific embodiments, which are disclosed and still obtain a like or similar result without departing from the invention.

### Example 1

### Preparation of CP Secretory Cell Implants

This example relates to the preparation of choroid plexus secretory cells suitable for encapsulation and implantation. All procedures are carried out in “GMP” licensed facilities, including strict infection barriers.

Neonatal pigs were anaesthetized with ketamine (500 mg/kg) and xylazine (0.15 mg/kg) and killed by exsanguination. The brain was immediately removed and dissected through the midline to reveal the fork of the choroid vessels. The choroid plexus was extracted and placed in Hanks Balanced Salt Solution (HBSS, 0-4° C.) supplemented with 2% human serum albumin. The tissue was chopped finely with scissors, allowed to settle and the supernatant removed. Collagenase (Liberase, Roche, 1.5 mg/mL, in 5 mL HBSS at 0-4° C.) was added and the chopped tissues mixed, allowed to sediment at unit gravity (1×g) and the supernatant was again removed. Collagenase (1.5 mg/mL, in 15 mL HBSS at 0-4° C.) was added and the preparation warmed to 37° C. and stirred for 15-20 minutes. The digested material was triturated gently with a 2-mL plastic Pasteur pipette and passed through a 200-μm stainless steel filter.

The resulting neonatal pig preparations were mixed with an equal volume of RPMI medium supplemented with 2-10% neonatal porcine serum (prepared at Diatranz/LCT). The preparations were centrifuged (500 rpm, 4° C. for 5 minutes), the supernatant removed and the pellet gently re-suspended in 30 mL RPMI supplemented with serum. This procedure produced a mixture of epithelioid leaflets or clusters of cells, about 50-200 microns in diameter, and blood cells. Blood cells were removed by allowing the mixture to sediment at unit gravity for 35 minutes at 0-4° C., removing the supernatant and re-suspending. The preparation was adjusted to approximately 3,000 clusters/mL in RPMI with 2-10% serum, and placed in non-adherent Petri dishes. Half of the media was removed and replaced with fresh media (5 mL) after 24 hours and again after 48 hours. By this time, most clusters assumed a spherical, ovoid or branched appearance.

The cells were then encapsulated in alginate as follows:

Encapsulation

A counted sample of choroid plexus clusters are washed twice in HBSS supplemented with 2% human serum albumin and once in normal saline. The majority of supernatant is removed from above the sedimented clusters and alginate (1.7%) added in the ratio 1 mL per 40,000 clusters. The clusters are carefully suspended in alginate and pumped through a precise aperture nozzle to produce droplets, which are displaced from the nozzle by either controlled air flow or by an electrostatic potential generated between the cell suspension exiting the nozzle and the receiving solution.

The stirred receiving solution contains sufficient calcium chloride to cause gelation of the droplets of alginate and cell cluster mixture. After the suspension has passed through the nozzle and the droplets collected in the calcium chloride solution, the gelled droplets are coated sequentially with poly-L-ornithine (0.1% for 10 min), poly-L-ornithine (0.05% for 6 min) and alginate (0.17% for 6 min). The gelled droplets are then treated with sodium citrate (55 mM for 2 min) to remove sufficient calcium from the interior of the gelled capsules to liquidize the contents. The poly-L-ornithine provides sufficient bonding for the capsule wall to remain stable.

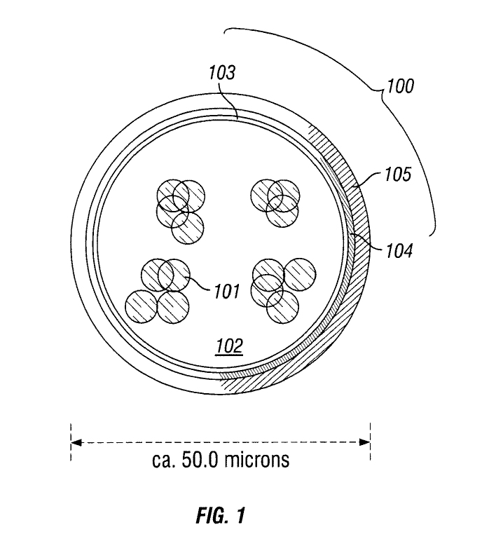

FIG. 1 shows a diagram of an encapsulated choroid plexus cluster 100. Clumps of cells 101 float in liquid 102 within a hollow sphere made up of several layers or coatings 103, 104, 105 which may be alternating alginate, polyornithine and alginate layers. Preferably, the innermost layer at least includes laminin.

The characteristics of the capsules thus produced are reproducibly of 530-670 microns in diameter (98-100%), are spherical (less than 2% are elliptical or otherwise misshapen). There are few broken capsules (less than 1%). Empty capsules, containing no CP clusters are typically less than 15%. The majority of the cell clusters within the capsules are 100-300 microns along their longest axis. Small clusters (less than 100 microns) are typically 5-13% and large clusters (greater than 300 microns along their longest axis) represent approximately 1-4% of the total.

After encapsulation the cell clusters were more than 90% viable as determined by Acridine Orange/Propidium Iodide staining.

Cryopreservation

Choroid plexus (CP) cell clusters and encapsulated CP were prepared as described above and maintained in culture for a minimum of 1-3 days. The CP cell clusters or encapsulated CP were sedimented, transferred to a cryo-vial, sedimented again and the supernatant removed. The CP cell clusters (20,000/mL) or encapsulated CP (10,000/mL) were suspended gently in porcine serum containing dimethyl sulphoxide (10%).

The suspended CP cell clusters or encapsulated CP were frozen at a rate of approximately 1° C. per minute in a freezer with a minimum temperature of −86° C. After 2 hours, the cryo-vials were placed in a liquid nitrogen storage facility.

Thawing the frozen suspensions was carried out by warming the cryo-vials in water at 37° C. As soon as the majority of the frozen medium was thawed, the suspension was diluted with a 20× volume of culture medium at room temperature, centrifuged at 15×g for 5 min, the supernatant removed and the pellet resupended in fresh culture medium.

The cryopreservation procedure described above resulted in recovery of live cells, in both free and encapsulated forms. The survival of the cells, as determined by acridine orange/propidium iodide staining was 80-97%. Surviving cells were also observed to show ciliary movement.

In Vitro Biological Activity

Choroid plexus (CP) cells secrete a cocktail of neurotrophic factors. In the following study, CP-conditioned media promoted the survival and function of fetal rat neuronal cells in culture, providing protection from neurotrophic deprivation in vitro.

In vitro biological activity of choroid plexus was determined by placing CP-conditioned media onto primary day 15 embryonic cortical neurons and measuring its effects on neuronal survival under serum deprivation conditions. The techniques used for preparing and maintaining primary cortical neuronal cultures were similar to those described previously (Fukuda A, Deshpande SB, Shimano Y, Nishino H. “Astrocytes are more vulnerable than neurons to cellular Ca²⁺ overload induced by a mitochondrial toxin, 3-nitropropionic acid.” Neuroscience, 87:497-507, 1998). Brains were removed from Wistar rats on embryonic day 15 and incubated in HBSS chilled on ice. The cortical tissues were dissected free, chopped into small pieces and incubated with Ca²⁺-free Hanks' solution containing trypsin (0.05 mg/mL) and collagenase (0.01 mg/mL) at 37° C. for 30 minutes, followed by the addition of soybean trypsin inhibitor (0.1 mg/mL) and DNase (0.1 mg/mL). The tissue was then centrifuged for 5 minutes (1000 rpm) in Dulbecco's modified Eagle's medium supplemented with 10% fetal bovine serum. The pellet was re-suspended and a homogenous cell suspension was made by gentle trituration using a fire-polished Pasteur pipette. Cells were plated on 35 mm tissue culture dishes (5×10⁴ cells/mL). The culture dishes were kept in a humidified incubator under 5% CO₂ and 95% air at 37° C. for 4 days. On day 4, cells were re-plated in 24-well plates, and over the next two days, a subset of cells were cultured without serum and with a range of concentrations of conditioned media (0-30%). CP-conditioned media was prepared as described in Example 3 herein, and stored at −20° C. prior to use. On day 6, cell viability was analyzed using Trypan blue exclusion. All studies were conducted in triplicate.

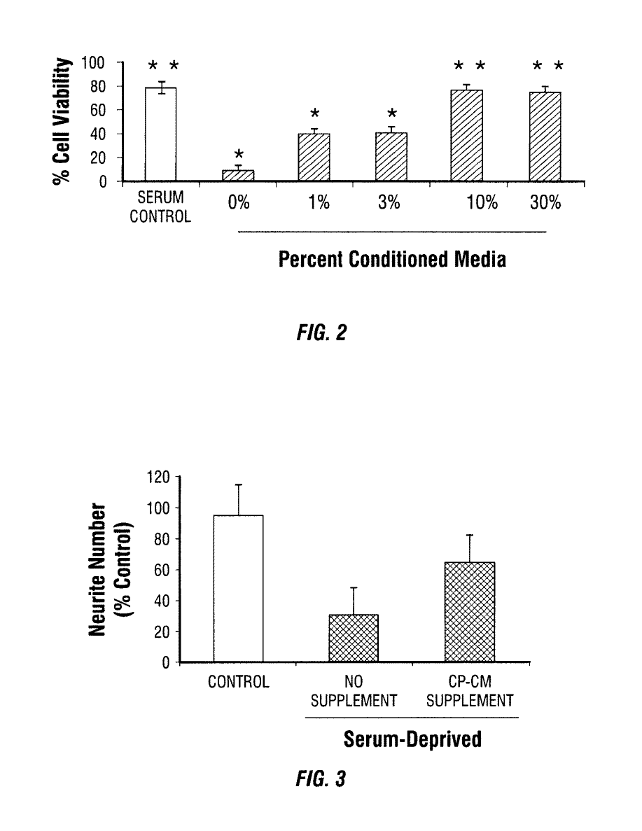

The results of these in vitro tests demonstrated that molecules secreted from the encapsulated choroid plexus exerted potent neurotrophic effects. An overall ANOVA revealed treatment effects on neuronal cell viability (ANOVA, F_{5,38}=109.01, p<0.0001). Primary cortical neurons deprived of serum for 2 days exhibited significant cell death (approximately 90%) compared to cells maintained in serum media (FIG. 2). Conditioned media collected from pig choroid plexus significantly protected against serum deprivation-induced cell death. This effect was dose-dependent with maximal effects obtained when serum-deprived neurons were cultured with 10% to 30% conditioned media from pig choroid plexus (p's<0.0001). At these concentrations, neuronal survival was 60%-85% and did not differ significantly from serum maintained cells (p's>0.05).

Neuronal Cell Function

Primary cultures of day 15 fetal rat brain cells were prepared according to the method described above, and placed in 96-well plates. The cultures were established for 4 days with Neurobasal medium supplemented with B27 nutrients and with fetal bovine serum (10%). After establishment, the cultured cells were divided into three groups: control cells grown in serum-supplemented media; cells grown in unsupplemented media; and cells grown in media supplemented with CP-conditioned media. The number of neurite processes and the degree of outgrowth of the neurite processes was then determined for each group of cells, as indicators of neuronal cell function.

Neurons cultured in Neurobasal medium supplemented with B27 nutrients and with fetal bovine serum (10%) became established and put out neurite extensions, the principle and essential components of neuronal networking. These neurite processes increased in number and length under the influence of neurotrophic growth factor present in serum.

When the medium was replaced with unsupplemented media so the neurons were without the support of neurotrophic growth factors in serum, the neurites decreased in number and length.

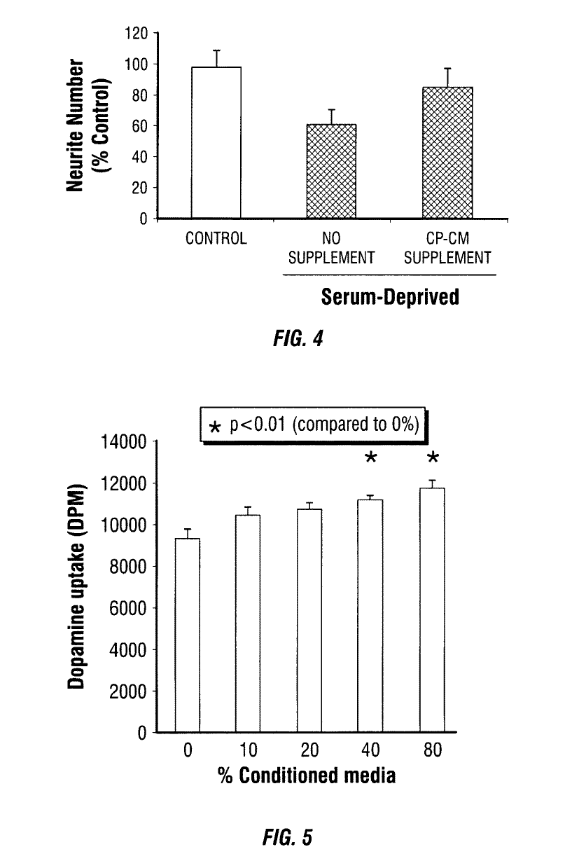

When the medium was instead supplemented by 50% with medium conditioned for 48 hrs by the secretory activity of choroid plexus cell clusters, the neurite numbers (CP-CM supplement, FIG. 3) and neurite extensions (CP-CM Supplement, FIG. 4) were significantly restored toward the numbers and length seen with 10% fetal bovine serum (FIG. 3 and FIG. 4, Control), demonstrating that choroid plexus cell clusters produce significant quantities of neurotrophic factors and growth factors capable of protecting neurons from serum deprivation-induced cell death and of enhancing neuronal cell survival and viability in culture. These CP produced factors are further capable of stimulating and/or maintaining the neuronal neurite processes essential for central nervous system networking that underpins all brain activity. Although the response to the CP-CM supplement does not exceed the response to serum, the protein concentration in CP-CM is less than 1% of that in the serum supplement and thus an extremely potent agent. These experiments demonstrate that choroid plexus-derived factors are able to enhance the survival and function of neuronal cells in vitro.

Biologically Active Factors in CP Clusters

Neonatal porcine and adult rat choroid plexus were isolated and grown in cell culture for 7-8 days with 5% CO₂ in air and RPMI supplemented with 2% neonatal porcine serum, nicotinamide and cyproxin as described above.

Cell clusters (approximately 2000-2500) were sedimented, media removed down to a volume of 100 microliters and the suspension mixed with 2% low-melting agarose in Hanks Balanced Salts solution (200 microliters) at 35-37° C. The material was allowed to cool and the solidified agarose block containing the cell clusters was fixed in neutral buffered formalin. The block was processed using standard procedures into paraffin wax and sections of this block were cut (5 microns thick) on a microtome and placed on standard glass microscope slides. The sections were stained for neu-N using an anti-serum specific for neu-N using an immunohistochemical detection technique according to the anti-serum supplier's instructions.

Approximately 20% of the cells, mostly at the periphery of the clusters, were found to be positive for the neuronal precursor marker neu-N, indicating that choroid plexus comprise cells with the characteristics of multipotent neuronal precursors, and that such cells survive and maintain their phenotype when cultured under the conditions described herein.

Assessment of Neurotrophic Bioassay Technique for Testing Conditioned Media from Processed Choroid Cells.

For this experiment cells of the Sks cell line (a neuroblastoma) were plated into a sterile 96-well plate at 10,000 cells per well and after 24 hours “settling” at 37° C. in humidified air, the original media was replaced by varying proportions of conditioned media as described above. Then the cells were visually assessed and scored according to the amount of growth, the number of dendrites, and connections overlapping other cells. The 50% conditioned media group grew dense connections and dendrites, which covered the whole well.

TABLE 1
Neuronal growth rate for a Sks cell line, scored after 36 hours
exposure to conditioned 10 media

| | 100% CM | 50% CM | 10% CM | 1% CM | control |
|---|---|---|---|---|---|
| Row 1 | +++ | ++++ | ++ | + | + |
| Row 2 | +++ | ++++ | ++ | + | + |
| Row 3 | ++++ | +++++ | ++ | + | + |
| Row 4 | +++ | ++++ | ++ | + | + |

A Dopamine Uptake Bioassay Technique for Testing Conditioned Media from Processed Choroid Cells (see FIG. 5).

For this experiment, mesencephalon (Mes) cells—which would include glia and neurones—were isolated from E15 rat fetuses and incubated in MEM plus 5% fetal bovine serum. Next day the cell media was aspirated, the wells were rinsed once with MEM, 400 microlitres of N2 media added (including selenium, progesterone, BSA, insulin and transferrin) and 600 microlitres of RPMI media was added, containing conditioned media, to a final concentration of 0, 20%, 40% and 60%. The RPMI includes 10 mM nicotinamide and 5% human serum albumen. The cells were incubated for 36 hours and then standard dopamine and GABA uptake assays were performed. The results are illustrated in FIG. 2, showing that dopamine uptake rises as the concentration of conditioned media is increased.

### Example 2

### Delivery of CP Cells

Thread-like single implants are for several reasons preferred over a loose suspension of globules containing cells. For example, if a single globule breaks, the cells within may be released with adverse consequences such as of immunological rejection, or transfer of latent virus infection that may be carried by the cells. Also, neurosurgeons are understood to prefer to use threads because they resemble existing tubular implants such as shunts, and drainage and monitoring catheters for use in the intracranial ventricles. Surgical techniques for the placement and later removal of these are well established. An ability to remove implants according to the invention is likely to be useful. A significant amount of medical technology exists (e.g., “Medtronic”, California) in relation to shunts, and drainage and monitoring catheters for use in the intracranial ventricles. Therefore, Example 2 comprises compatible objects for the delivery of xenotransplants in the form of living cells within selectively permeable tubing. Such tubing comprises a kind of disposable, implantable device that carries either an inner surface lining of a laminin in order to induce the cells to settle, or includes a cavity holding protected choroid plexus cells as described above, which can be left in place for a long period of perhaps months.

All these systems involve the usual precautions (sterility and care) needed for an intracranial operation; however it may be possible to perform it under a local instead of a general anaesthetic, and hence the procedure is more compatible with use in developing countries, and/or where costs should be minimized.

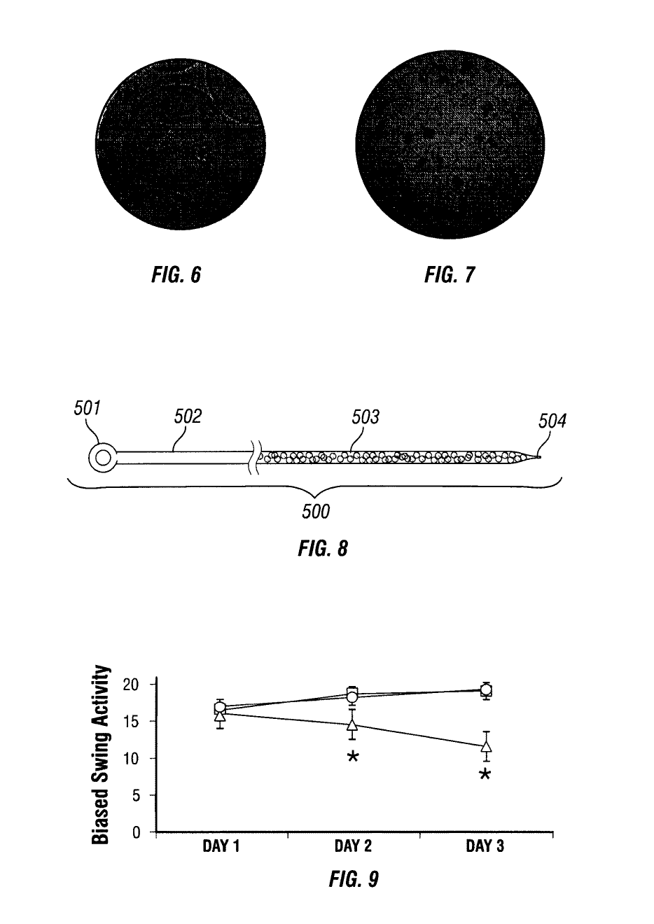

Surgical techniques for the implantation of a composition according to the invention can be carried out, preferably into a lateral ventricle, and preferably from a frontal, parietal, or occipital approach. The occipital approach, being more or less in line with the long axis of a lateral ventricle, allows a longer “artificial choroid” to be deposited. (It should be noted that in many of the conditions considered as appropriate for this type of treatment, stereotaxic techniques are difficult if not impossible owing to the brain becoming distorted). An example artificial choroid may comprise one or a bundle of dialysis-type hollow fibres containing free cells. Optionally, the fibre may be a tougher, more porous device (like a teabag) holding associations of coated cells or globular capsules, and in that case the permeability requirement is conveniently a function of the encapsulation rather than of the fibre which can be stronger. Preferably the fibre has an active end, and an inactive end by means of which the implant may later be retrieved. FIG. 8 shows at 500 such a fibre. The inactive end 502 includes an eyelet 501 and the active end is heat-sealed 504. The permeable portion of the fibre includes globular capsules 503 holding active cells (see FIG. 6 or FIG. 7).

Implants may be distributed for use within a container holding a conventional liquid lifesupport media as is well known in the art. A kit of materials for surgical implantation of an implant may also be distributed in order to facilitate a sterile, slow virus-free operation. A minimal kit of materials might include a blade to guide a cannula into the ventricle, a cannula, and a container holding implants. A more comprehensive kit would also include drapes, skin preparation materials, scalpels, haemostats, a drill for obtaining surgical access through the cranium to the central nervous system, a blade guide, sutures, and dressings.

### Example 3

### Source of Cells

Selection of choroid plexus cells capable of expressing a given balance of trophic factors, to use the term in a broad sense, is preferably done by selecting a particular species of mammal, and age of mammal from which the cells are to be harvested so that the cells of its choroid plexus already function as required. The age may be anywhere from perhaps mid-gestation or before, when a choroid plexus is identifiable, to somewhere in postnatal life. The output of the choroid plexus—in terms of trophic factors—changes during development of the brain through gestation and for perhaps a year afterwards. Myelination, for example, continues to proceed well after birth. Accordingly, modification of harvested cells in order to manipulate their properties as by restriction of the environment or by introduction of genetic material (DNA) is not expected. However, there may be instances when such steps are indicated. (Restriction, such as measures to adapt the cells to function within a relatively low PO₂, is already provided for in this invention because a fetus has a lower PO₂ in general). This invention may also be applied to cells taken from a human source. It may be possible to construct genetically modified and coated/protected choroid plexus cells for use in an xenotransplant designed for the purpose of compensating for a disease wherein inborn errors of metabolism affect the CNS wherein the implanted cells metabolise and thereby consume undesirable compounds, or compensate with products for other cells in the brain that fail to secrete desirable compounds, on the brain side of the “blood-brain barrier”. This may be useful in lysosomal storage diseases or for other genetically based defects such as aspartoacyclase deficiency.

### Example 4

### Artificial Organ

This Example describes the use of a vascularized mechanical construction at least partially simulating the architecture of a choroid plexus; as a two-compartment form of “artificial organ.” It may be located elsewhere in the body at a convenient, preferably subcutaneous or intraperitoneal site and surgically anastomosed between an artery and a vein. A tube, like a shunt as used for hydrocephalus, is connected between the artificial organ and a ventricle or the like, to carry the cerebrospinal fluid-like output from the device into the central nervous system. The usefulness of this approach is in part based on the possibility that a relatively large volume of choroid plexus material, well vascularized, may be required to supply adequate amounts of both CSF fluid and trophic material for some neurological diseases.

Given that in nature the choroid plexus overlies an array of capillaries evidently exuding fluid, it may be useful to construct an implantable module including an artificial semi-permeable filter element exposed on one side to a flow of blood at an effective pressure, having on the other side an accumulation of choroid plexus epithelial cells optionally attached by means of an artificial basement membrane including a laminin or the like to be washed with the transudate, and a conduit for the transudate plus growth factors leading to the ventricular system of the brain. Preferably a filter is included in the outflow so that cellular material is not swept into the ventricle. It may be possible to either construct, or to cause the cells used in the artificial organ to mimic the extensively folded nature of the actual choroid plexus. In practice, there may be some usefulness in minimizing a possible release of angiogenic factors backward from the active cells into the draining blood if the rate of flow is small, and usefulness in providing protection against an excessive quantity of fluid passing from the organ into the ventricle. In a worst case, the fluid might comprise blood. An active control device, also capable of receiving external commands from time to time, may be included in the artificial organ and a precedent in life for such control means is the known extensive innervation received both by the vasculature and by the secretory cells of the choroid plexus.

### Example 5

### Rodent Model of Stroke

This example describes the use of neonatal porcine choroids plexus (CP) cells implanted into the brain of rats which have undergone stroke surgery to assess the effect of the CP implant on neurological function. All procedures in this and the foregoing examples were carried out following NIH and Society for Neuroscience guidelines for use of animals in research and all surgical procedures were conducted under aseptic conditions.

Neonatal porcine choroid plexus cells were prepared and encapsulated as described above in Example 1.

Stroke Surgery

Adult male Wistar rats (supplied by University of Auckland, NZ) approximately 3 months of age and weighing 250-350 grams served as subjects. Animals were housed in a temperature (22±1° C.) and humidity (50±5%) controlled environment and had free access to food and water throughout the study, except for 4 hours prior to surgery.

Rats were anesthetized using equithesin (300 mg/kg i.p.). Permanent unilateral focal neocortical ischemia was produced using a well-established middle cerebral artery (MCA) occlusion/reperfusion model. Based on our previous studies and those of several other laboratories (Borlongan C. V., Cahill D. W., Sanberg P. R. “Locomotor and passive avoidance deficits following occlusion of the middle cerebral artery.” Physiol. Behav., 58:909-17, 1995a; Borlongan C. V., Sanberg P. R. “Elevated body swing test: a new behavioural parameter for rats with 6-hydroxydopamine-induced hemiparkinsonism.” J. Neurosci., 15:5372-8, 1995c), a one-hour occlusion of the MCA was used to produce a maximal infarction. Briefly, an incision was made to expose the right MCA and a nylon suture (length=15-17 mm; tip diameter=24-26 gauge) was inserted to completely occlude the MCA. After a one-hour occlusion, the suture was removed and the incision closed using routine procedures. Body temperature and blood gases of animals undergoing such surgical procedure remained within normal limits.

CP Transplantation Surgery

Immediately following MCA occlusion (i.e. within 10 minutes) animals were placed in a stereotaxic apparatus (Kopf Instruments). A craniotomy (2 mm wide×3 mm in length) was performed over the predicted core of the cerebral infarction using a surgical microdrill. The coordinates for the craniotomy were: ML=3.0 mm to 5.0 mm and AP=+1.0 mm to −2.0 mm—from Bregma (Paxinos, G. and C. Watson. “The Rat Brain in Stereotaxic Coordinates”, Academic Press, New York, 1986). For transplantation, the dura was excised and 50-55 hand-picked microcapsules were suspended in 30 μL of isotonic saline and placed into the previously formed craniotomy. The excess saline was gently removed resulting in a bed of alginate capsules overlying the cortex. To help maintain the positioning of the capsules, a small piece of collagen was placed over the capsules and the incision sutured closed. Animals were then placed on a temperature-controlled pad until recovery from anesthesia. These procedures resulted in the formation of 3 experimental groups: (1) Stroke only (MCA+craniotomy but no transplant; n=10), (2) Stroke+control transplant (empty capsules; n=10) and (3) Stroke+choroid plexus loaded capsules; n=11).

Behavioural Testing

Motor Asymmetry

Because motor asymmetry (i.e., bias movements to one side of the body) is consistently displayed by MCA-occluded rats (Borlongan C. V., Cahill D. W., Sanberg P. R., “Locomotor and passive avoidance deficits following occlusion of the middle cerebral artery.” Physiol. Behay., 58:909-17, 1995a; Borlongan C. V., Martinez R., Shytle R. D., Freeman T. B., Cahill D. W., Sanberg P. R., “Striatal dopamine-mediated motor behaviour is altered following occlusion of the middle cerebral artery.” Pharmacol. Biochem. Behay., 52:225-9, 1995b) the elevated body swing test (EBST) was used to confirm the functional consequences of the MCA occlusion and to quantify improvements in motor function produced by the choroid plexus transplants. Animals were tested daily on days 1, 2, and 3 post-surgery. The EBST is known to reliably detect stable motor asymmetry at these early time points. Individual animals were gently picked up at the base of the tail and elevated until the animal's nose was at a height of 5 cm above the test surface. The direction of the swing, either left or right, was counted once the animals head moved sideways approximately 10 degrees from the midline position of the body. After a single test, the animal was lowered and allowed to move freely for 30 seconds prior to retesting. These steps were repeated 20 times for each animal. The results are shown in FIG. 9.

Neurological Evaluation

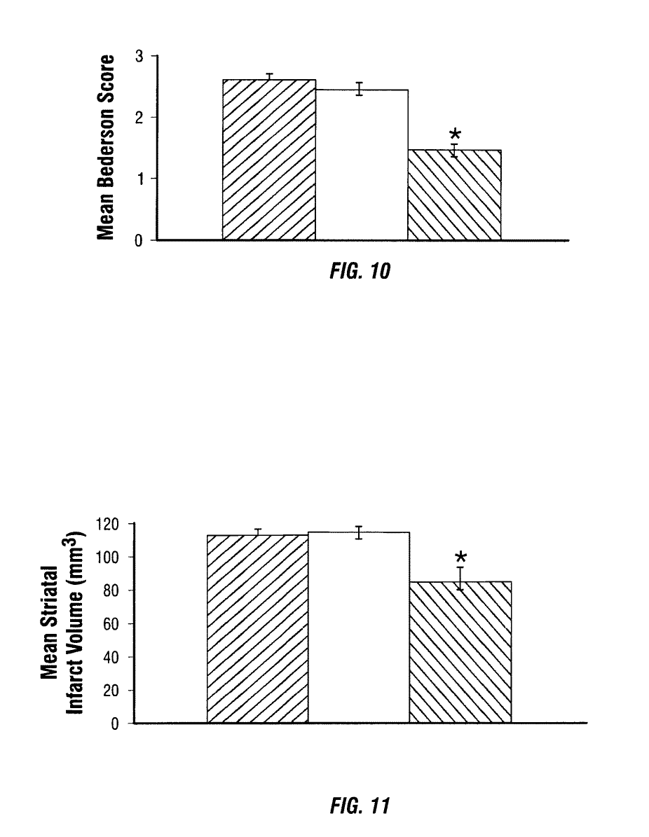

Animals were tested for neurological function using the conventional (Bederson tests) at three days post surgery. A neurologic score for each rat was obtained using 3 tests that included (1) contralateral hindlimb retraction that measured the ability of the animal to replace the hindlimb after it was displaced laterally by 2 to 3 cm, graded from 0 (immediate replacement) to 3 (replacement after minutes or no replacement); (2) beam walking ability graded 0 for a rat that readily traversed a 2.5-cm-wide, 80-cm-long beam to 3 for a rat unable to stay on the beam for 10 seconds; and (3) bilateral forepaw grasp that measured the ability to hold onto a 2-cm-diameter wooden rod, graded 0 for a rat with normal forepaw grasping behaviour to 3 for a rat unable to grasp with the forepaws. The scores from all 3 tests were conducted over a period of approximately 15 minutes and were combined to give an average neurologic deficit score (total score divided by three). The results are shown in FIG. 10.

Histology

Following behavioural testing on day 3 post-stroke, animals were anesthetized with lethal dose of equithesin (500 mg/kg, i.p.), perfused with 100 mL of ice-cold saline, decapitated and the brains harvested. To confirm viability of the transplanted cells the capsules were flushed from the transplant site using sterile saline. Quantitative histological determinations of infarct volume were performed using standard TTC staining and quantitative image analysis as previously described (Wang Y., Chang C. F., Morates M., Chang Y. H., Hoffer J. “Protective Effects of Glial cells line-derived neurotrophic factor in ischemic brain injury”. Ann. N.Y. Acad. Science, 962:423-37, 2002). Infarct volume was determined using the following formula=2 mm (thickness of the slice)×[sum of the infarction area in all brain slices (mm²)]. The results are shown in FIG. 11.

Results

Behavioural Testing

Choroid Plexus Grafts Reduce Stroke-Induced Motor Deficits

As shown in FIG. 6, choroid plexus transplants significantly reduced the motor asymmetry produced by MCA occlusion. An overall ANOVA revealed significant treatment effects over the 3 day post-stroke period (F_{2,90}=28.07, p<0.0001). While a trend towards improved performance was seen in those animals receiving choroid plexus transplants as early as 1 day post-surgery this benefit was modest and did not reach statistical significance (p>0.05). Bonferroni's post-hoc t-tests did, however, demonstrate that stroke animals receiving choroid plexus transplants (▴) displayed significant ameliorations of motor asymmetry (FIG. 9) at days 2 and 3 post-surgery (>16% and >23%, respectively) compared to control animals (empty capsules (▪) or stroke only ◯); p's<0.01). These reductions translated to an average motor asymmetry of 74% and 62%, which are below the conventionally accepted 75% criterion for MCA-occluded rats to be considered significantly impaired on this test. No significant changes were noted in either control group throughout testing.

Choroid Plexus Grafts Reduce Neurological Deficits

Similar benefits of encapsulated choroid plexus transplants were observed on neurologic impairment. Animals were tested for neurological function on day three post-surgery using the Bederson test (ANOVA, F_{2,28}=50.6, p<0.0001) (FIG. 10). Post-hoc comparisons demonstrated that while MCA occlusion produced pronounced deficits in performance in control animals, stroke animals that received choroid plexus transplants exhibited significant improvements in neurological performance. Those animals receiving choroid plexus transplants were improved by 35%-40% relative to the control animals (p's<0.0001). There were no detectable differences in performance between the control groups at any time or on any test (p>0.10).

Histology

Choroid Plexus Grafts Reduce Stroke-Induced Cerebral Infarcts

Three days following MCA occlusion and transplantation, the volume of cerebral infarct was determined in all animals using TTC staining and quantitative image analysis. Consistent with previous studies, MCA occlusion produced a large cerebral infarct that encompassed much of the striatum in control animals. The attenuation of behavioural deficits in stroke animals receiving choroid plexus transplants was accompanied by a significant reduction in cerebral infarction (ANOVA, F_{2,28}=4.77, p<0.05). Relative to control animals the volume of striatal infarct was significantly reduced by about 30% (FIG. 11; p's<0.05).

### DISCUSSION

This in vivo experiment demonstrates the neuroprotective effects of choroid plexus cells on neurons that are otherwise destined to die. The transplanted choroid plexus significantly reduced the extent of cerebral infarction and motor/neurological deficits following MCA occlusion in rats. This study did not attempt to optimize the transplant site or the numbers of cells used per recipient. Rather, based on previous studies, the cell-loaded capsules were simply placed on the cortex overlying the striatal region that would be normally infarcted following MCA occlusion. Without wishing to be bound by theory, it is believed that this paradigm provided a fairly stringent test of the ability of the molecules secreted from the choroid plexus to exert a neuroprotective effect since the molecules would be required to diffuse from the capsules and through several mm of cortical tissue. Accordingly, the concentrations of therapeutic molecules reaching the infarcted region would be modest compared to those achieved locally. Nonetheless, even under these less than ideal conditions, a significant structural and functional benefit was produced by the choroid plexus transplants.

The use of alginate microcapsules to encapsulate the choroid plexus in this study provided the advantages of eliminating the need for chronic immunosuppression of the host and allowing the implanted cells to be obtained from xenogeneic sources (i.e., porcine cells used in the current studies) thus avoiding the constraints associated with cell sourcing. These microcapsules conferred the additional advantage of facilitating transplantation and localization on the cerebral cortex in the current studies.

### Example 6

### Rodent Choroid Plexus Cells in Rodent Model of Huntington's Disease

This example describes the use of encapsulated rat choroids plexus (CP) cells transplanted into the brain of rats which had undergone surgery to produce a Huntington's disease model, to assess the effect of the autologous implant on neurological function.

Animals

Adult male Wistar rats (supplied by University of Auckland, NZ) approximately 3 months of age and weighing 250-350 grams served as subjects. Animals were housed in a temperature (22±1° C.) and humidity (50±5%) controlled environment and had free access to food and water throughout the study, except for 4 hours prior to surgery.

Isolation, Culture, and Encapsulation of Rat Choroid Plexus Cells

Adult Wistar rats (supplied by University of Auckland, New Zealand) were anaesthetized with ketamine (500 mg/kg) and xylazine (0.15 mg/kg) and killed by exsanguination. The brain was immediately removed, and the CP was extracted and placed in Hanks Balanced Salt Solution (HBSS, 0-4° C.) supplemented with 2% human serum albumin. The tissue was minced and the supernatant removed. Collagenase (Liberase, Roche, 1.5 mg/mL, in 5 ml HBSS at 0-4° C.) was added and the chopped tissues mixed, allowed to sediment at unit gravity (1×g), and the supernatant was again removed. Collagenase (1.5 mg/mL, in 15 mL HBSS at 0-4° C.) was added and the preparation warmed to 37° C. and stirred for 15-20 minutes. The digested material was triturated gently with a 2-mL plastic Pasteur pipette and passed through a 200 μm stainless steel filter. The resulting CP preparations were mixed with an equal volume of RPMI medium supplemented with 10% Fetal Bovine Serum. The preparations were centrifuged (500 rpm, 4° C. for 5 minutes), the supernatant removed, and the pellet gently re-suspended in 30 ml RPMI supplemented with serum. The preparation was adjusted to approximately 3,000 clusters/ml in RPMI with 10% Fetal Bovine Serum and placed in non-adherent Petri dishes. Media was replenished after 24 and 48 hours. Prior to encapsulation, the cell clusters were washed by sedimenting 3 times in 2% human serum albumin in HBSS (30 mL) at room temperature. The cells were then encapsulated in alginate according to previous published protocols. Encapsulated cells were maintained in culture for 7 days prior to transplantation.

Surgery

Immediately prior to surgery, rats were anesthetized with equithesin (300 mg/kg; i.p.) and positioned in a stereotaxic instrument (Kopf, Tujunga, Calif., USA). A midline incision was made in the scalp and a hole drilled through the skull for placement of cell-loaded alginate capsules into the striatum using an 18-gauge Teflon™ catheter mounted to the stereotaxic frame. The stereotaxic coordinates for implantation were: 0.5 mm anterior to Bregma, 1.5 mm lateral to the sagittal suture, and 7.5 mm below the cortical surface (Paxinos and Watson, 1980). Ten empty or cell-loaded capsules were injected into the striatum of Wistar rats (n=9 per group, 3-months old and approximately 300 grams) using an 18-gauge Teflon™ catheter mounted to a stereotaxic frame (Kopf Instruments). An additional control group of animals (n=8) received no capsules, only vehicle infusion. Following implantation, the skin was sutured closed.

Three days following implantation of the capsules, all animals were anesthetized, placed into the stereotaxic instrument, and unilaterally injected with 225 nmol of QA (Sigma) into the striatum at the following coordinates: 1.2 mm anterior to Bregma, 2.6 mm lateral to the sagittal suture, and 5.5 mm ventral to the surface of the brain. QA was infused into the striatum using a 28-gauge Hamilton syringe in a volume of 1 μL over 5 minutes. The injection cannula was left in place for an additional 2 minutes to allow the QA to diffuse from the needle tip, after which the cannula was removed, and the skin sutured closed. Immediately following the QA lesion, animals were injected i.p. with 10 mL of a lactated Ringer's solution. At 28 days post capsule implantation, animals were anesthetized and decapitated and the brains processed for histology.

Behavioral Testing

To quantify potential sensory neglect, the forelimb placing test was used to test the animal's ability to make directed forelimb movements in response to sensory stimuli. Rats were held so that their limbs were hanging unsupported and the length of their body was parallel to the surface of a stainless steel table. They were raised to the side of the table so that their whiskers made contact with the top surface on 10 trials for each forelimb. Unilaterally lesioned animals have been shown to display impairment in placement of their contralateral (to the lesion side) limb. Rats were given one trial at 14 days and 28 days post-lesion. To further quantify the functional effects of the QA lesion and any potential benefits of the CP transplants, the body weights of all animals were recorded every 2-3 days.

Histology

At the conclusion of behavioral testing the animals were anesthetized and transcardially perfused using 100 mL of saline (4° C.) followed by 250 mL of 4% paraformaldehyde. All solutions were ice-cold (4° C.). Brains were removed after fixation, and refrigerated for approximately 48 hours prior to being placed in 25% buffered sucrose (pH 7.4). Sections throughout the entire striatum were cut at 40 μm intervals on a cryostat and stored in a cryoprotectant solution. Adjacent sections through the striatum were processed immunocytochemically for choline acetytransferase (ChAT, 1:1000, Chemicon) using previously published procedures. All immunohistochemical reactions were terminated by three 1-minute rinses in PBS. Sections were mounted, dehydrated, and coverslipped. Control sections were processed in an identical manner except that the primary antibody or an irrelevant IgG was substituted for the primary antibody. A separate series of sections through the striatum were stained for NADPH-diaphorase (NADPH-d, Sigma) as described previously (Beal et al., Synapse, 3(1):38-47, 1989). Adjacent sections were stained for Nissl to aid in cytoarchitechtonic delineation and for quantitiative determination of lesion volume (see below).

Quantitative Morphometric Analysis

The volume of the lesion produced by QA was determined in all using a semi-automated image analysis system (NIH Image) as previously described. Sections spaced 240 μm apart that encompassed the entire lesion area were analyzed. The border of the lesion was traced on Nissl-stained sections, and the volume of the lesioned area in each animal was expressed in each animal in cubic millimeters. Using adjacent sections throughout the lesioned area, every sixth section was processed immunocytochemically for CHAT or diaphorase. The numbers of ChAT- and diaphorase-positive neurons were quantified throughout both the intact and lesioned striatum as previously described.

Statistics

Because no statistical differences were obtained on any measure between animals receiving QA alone or QA plus empty capsules, these groups were combined to form a single “control” group to facilitate graphical and statistical comparisons. For the behavioural placement test, repeated measures of ANOVA was initially conducted to show interaction effects between treatment (control versus CP grafts) and time (14 versus 28 days post-lesion), then Bonferroni compromised t-tests were used to reveal differences between treatment groups. For lesion volume and ChAT and NADPH cell counts, Student's t-tests were used to reveal differences between treatment groups.

Results

General Observations

No overt signs of behavioural or neurological toxicity were observed in any animals after implantation of either empty or CP-loaded capsules. During the postoperative recovery period after QA injections, the control animals exhibited whole-body barrel rotations that persisted for 2-4 hours. These same animals had a transient period of weight loss, piloerection, and diarrhea that subsided within several days after QA (Table 2). Animals that received QA together with CP capsules did not show whole-body rotations but did exhibit a slight motor asymmetry after QA. This asymmetry was transient, and recovery was seen within several hours. Animals that received CP before QA showed continued weight gain as compared to a small but significant weight loss in the group receiving QA alone (Table 2). No additional signs of systemic toxicity were noted.

TABLE 2
Cell Counts and Weight Loss in QA-Lesioned Rats Cell Counts

| | | Lesioned/implanted | | |
|---|---|---|---|---|
| | Intact side | side | | Percent loss |
| Lesion Volume | | | | |
| Control | no lesion | 6.3 ± 0.05 | mm³ | NA |
| CP | no lesion | 1.1 ± 0.06 | mm³ | NA |
| ChAT-positive cells | | | | |
| Control | 985 ± 61 | 604 ± 30 | | 39% |
| CP | 1159 ± 48 | 935 ± 27 | | 19% |

Body Weights
Day post QA (% pre-surgery)

| | 1 | 3 | 5 | 7 | 24 |
|---|---|---|---|---|---|
| Control | 105.13 | 97.59 | 95.9 | 97.94 | 104.29 |
| CP | 106.54 | 110.18 | 115.91 | 123.97 | 131.39 |

Behavioural Testing

Intrastriatal injections of QA produced significant performance deficits in the placement, bracing, and akinesia tests. This was evidenced by a decrease of 90% in the number of contralateral placements taken relative to the unimpaired ipsilateral limb in control animals. In contrast, a marked behavioural protection was observed when encapsulated CP cells were implanted immediately adjacent to the QA-lesioned striatum. ANOVA revealed significant main treatment effects (F_{1 24}=106.32, p<0.0001).

Relative to the normal limb, performance of the impaired limb was completely normalized as assessed using this measure. Animals that received CP grafts significantly performed better than controls at both 14 and 28 days post-lesion test periods (p's<0.0001).

Histology

Nissl-stained sections confirmed that the empty and CP-loaded capsules were consistently located within the striatum. The capsules were localized to the injection tract and were well tolerated within the host brain. There was little evidence of trauma as a result of surgery, and the inclusion of cells within the capsules did not result in any overt deleterious host tissue reaction. The surrounding host tissue produced slight deformation of the capsules, but there was no evidence that any capsules broke or degraded significantly during the course of the experiment. Capsules contained viable clusters of CP one month after transplantation without any evidence of significant cell death.

Within the host striatum, QA induced a characteristic lesion of intrinsic neurons. In agreement with previous studies (Emerich et al., J. Neurosci., 16:5168-5181, 1996; Kordower et al., Exp. Neural., 159:4-20, 1999), QA administration produced a substantial atrophy of the striatum, resulting in a marked ventricular dilation and cavitation of the overlying cortex. In animals receiving QA alone or empty capsules, the QA-induced lesion was elliptical in shape and encompassed much of the striatum at the level of the injection. The core of the lesion was filled with glial cells and scattered viable Nissl-stained cells. Many remaining neurons were shrunken and displayed a dystrophic morphology. In contrast, the size of the lesion was significantly reduced by 82% (p<0.0001) in those animals receiving CP implants compared with control animals (6.3±0.05 versus 1.1±0.06 mm³).

Cell counts were performed to quantify the extent of cell loss produced by QA and the subsequent protection mediated by CP transplants. It is important to note that the lack of immunolabeling does not necessarily equal a loss of neurons and the loss of ChAT- and NADPH-d-positive neurons refers to a loss of immunolabeling. ChAT- and NADPH-d-stained sections within the intact contralateral striatum revealed a general pattern of labeled perikarya consistent with previous reports (Emerich et al., J. Neurosci., 16:5168-5181, 1996; Kordower et al., Exp. Neurol., 159:4-20, 1999). Qualitatively, the QA lesion resulted in a dramatic loss of ChAT neurons within the striatum similar to that previously reported (Emerich et al., J. Neurosci., 16:5168-5181, 1996; Kordower et al., Exp. Neurol., 159:4-20, 1999). In sections proximal to the needle tract, there was almost a complete loss of ChAT-positive neurons. Those few neurons that did remain appeared atrophic with a stunted dendritic morphology. In contrast, there were numerous ChAT-positive neurons within the striatum of QA-injected rats that received CP transplants. Even in sections that contained the needle tract, many ChAT-positive cells were seen. These cells were large in size (25-35 μm in diameter) with long neuritic processes. They displayed the typical morphological profile of healthy cholinergic striatal interneurons. Quantitatively, the number of ChAT-positive cells was reduced on the lesioned side relative to the non-lesioned side (p<0.0001). This loss of ChAT-positive cells was significantly attenuated in rats receiving rat CP (19%) compared to control animals (39%).

### DISCUSSION

These experiments demonstrate that encapsulated CP can prevent the anatomical and behavioural sequelae seen in an animal model of HD.

### Example 7

### Neonatal Porcine Choroid Plexus Cells in Rodent Model of Huntington's Disease

This example describes the use of neonatal porcine choroid plexus (CP) cells transplanted into the brain of rats which have undergone surgery to produce a Huntington's disease model, to assess the effects of the CP xenograft on neurological function.

Transplantation of Neonatal Porcine CP

Neonatal porcine CP was isolated (Large White/Landrace cross, both sexes, 7-10 days old and 3.5 to 5.5 kg) using previously described protocols (Borlongan C. V., Skinner S. J., Geaney M., Vasconcellos A. V., Elliott R. B., Emerich D. F. “Intercerebral transplantation of porcine choroid plexus provides structural and functional neuroprotection in a rodent model of stroke.” Stroke. 35(9):2206-10, 2004). CP was maintained as approximately 3,000 epithelioid clusters/ml in RPMI in non-adherent Petri dishes. The cells were encapsulated in alginate as previously described (Calafiore et al., Transplant Proc., 4:2126-2167, 1997; Elliott et al., Cell Transpl., 9:895-901, 2000). Empty capsules were processed identically and encapsulated cells were maintained in vitro for 7 days prior to transplantation.

Adult male Wistar rats were anesthetized with equithesin (300 mg/kg; i.p.) and positioned in a stereotaxic instrument (Kopf, Tujunga, Calif., USA). A midline incision was made in the scalp and a hole drilled through the skull for placement of ten empty or ten CP cell-loaded alginate capsules into the striatum using an 18-Gauge Teflon™ catheter mounted to the stereotaxic frame. The stereotaxic coordinates for implantation were: 0.5 mm anterior to Bregma, 1.5 mm lateral to the sagittal suture, and 7.5 mm below the cortical surface (Paxinos and Watson, 1980). Ten empty (control) or cell-loaded capsules were injected into the striatum of each rat using an 18-Gauge Teflon™ catheter mounted to a stereotaxic frame (Kopf Instruments). A second control group of rats received no capsules, only vehicle infusion. Following implantation, the skin was sutured closed.

Surgery

Three days following implantation of the capsules, all animals were anesthetized, placed into the stereotaxic instrument, and unilaterally injected with 225 nmol of Quinolinic acid QA (Sigma) into the striatum at the following coordinates: 1.2 mm anterior to Bregma, 2.6 mm lateral to the sagittal suture, and 5.5 mm ventral to the surface of the brain. QA was infused into the striatum using a 28-gauge Hamilton syringe in a volume of 1 μL over 5 minutes. The injection cannula was left in place for an additional 2 minutes to allow the QA to diffuse from the needle tip, after which the cannula was removed, and the skin sutured closed. Immediately following the QA lesion, animals were injected i.p. with 10 mL of a lactated Ringer's solution. At 28 days post-capsule implantation, animals were anesthetized and decapitated and the brains processed for histology.

Behavioural Testing

To quantify the functional effects of the QA lesion and potential benefits of the CP transplants, the body weights of all animals were recorded every 2-3 days and sensory neglect was measured using the placement test as previously described (Salzberg-Brenhouse et al., J. Pharmacol. Exper. Therap., 306:218-228, 2003). For this test, each animal received 10 trials for each forelimb 14 days post-lesion.

Histology

At the conclusion of behavioural testing (6 weeks post-QA), the paraformaldehyde-preserved brains of all animals were removed and sections throughout the striatum were cut at 40 μm intervals on a cryostat. Adjacent sections through the striatum were processed for immunocytochemically for choline acetyltransferase (ChAT) and NADPH-diaphorase as previously described.

Results

General Observations

No overt behavioural or neurological toxicity resulted from implanting the empty or CP-loaded capsules. Control animals exhibited whole-body barrel rotations for 2-4 hours post QA together with a period of weight loss that subsided within several days. Animals receiving QA plus CP transplants did not show whole-body rotations or weight-loss, post-surgery in essentially the same way as those treated with rat CP (cf Table 3).

TABLE 3
Cell Counts and Weight Loss in Quinolinic Acid-Lesioned Rats Cell
Counts

| | Intact side | Lesioned/implanted side | | Percent loss |
|---|---|---|---|---|
| Lesion Volume | | | | |
| Control | no lesion | 6.18 ± 0.07 | mm³ | NA |
| CP | no lesion | 0.84 ± 0.02 | mm³(*) | NA |
| ChAT-positive cells | | | | |
| Control | 995 ± 48 | 630 ± 37 | | 37% |
| CP | 985 ± 55 | 816 ± 42(*) | | 17% |
| NADPH-d-positive | | | | |
| cells | | | | |
| Control | 750 ± 39 | 116 ± 19 | | 84% |
| CP | 790 ± 46 | 119 ± 22 | | 85% |

(*)Statistically significant difference, p < 0.05, between control and
choroid plexus.

Body Weights
Day post QA (% pre-surgery)

| | 1 | 3 | 5 | 7 | 24 |
|---|---|---|---|---|---|
| Control | 105.13 | 97.59 | 95.9 | 97.94 | 104.29 |
| CP | 106.54. | 110.18 | 115.91 | 123.97 | 131.39 |

Behavioural Testing

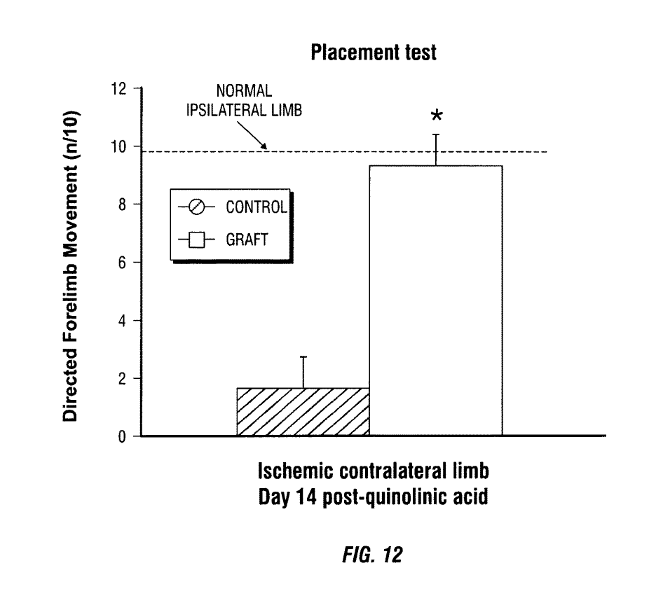

Further evidence of the functional benefits of the CP transplants was observed using the placement test. While performance with the intact ipsilateral limb was nearly perfect in all animals (range=9.6-9.8 out of 10 responses), the control animals were markedly impaired when using the contralateral limb (1.6±1.1 out of 10, see FIG. 12). Performance of the impaired limb was completely normalized in animals receiving CP transplants (9.2±1.3 out of 10); ANOVA F_{1,25}=4356.38, p<0.0001).

Histology

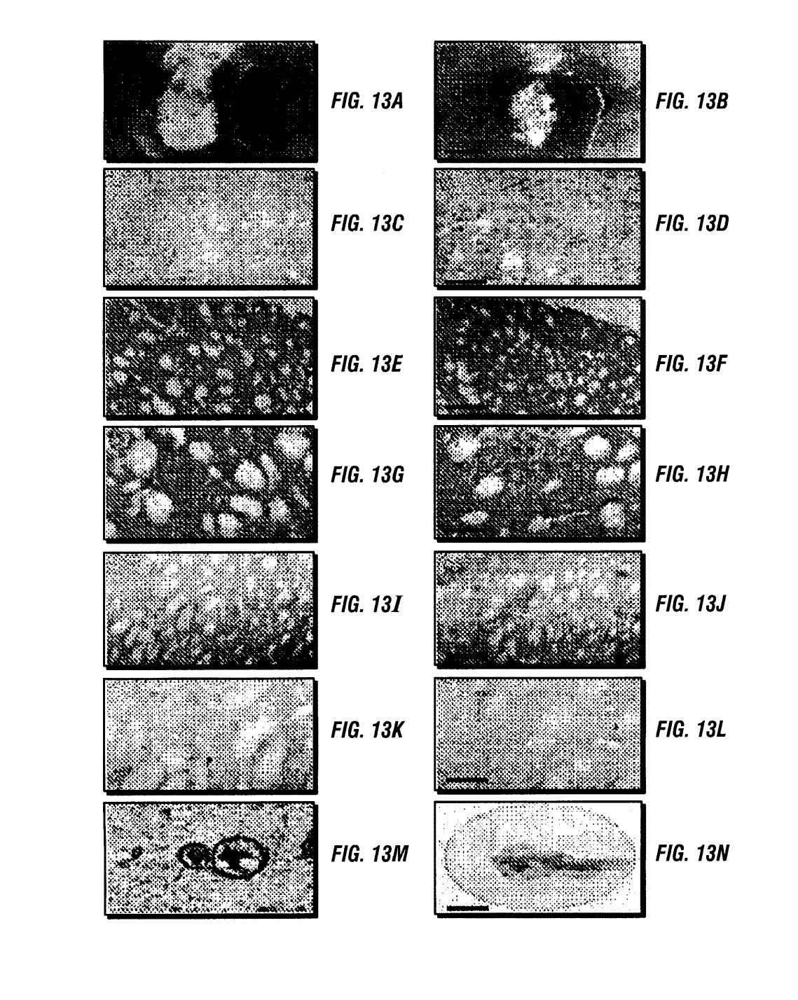

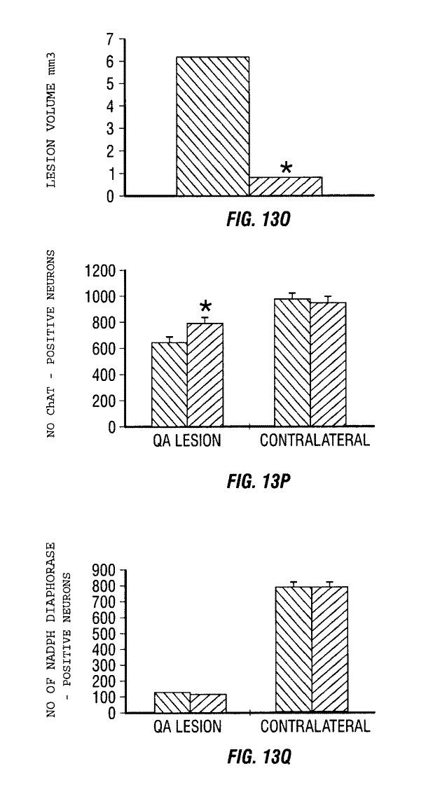

Empty and CP-loaded capsules were localized to the injection tract and were well tolerated within the host brain. Capsules contained viable clusters of CP 6 weeks after transplantation without any evidence of cell death. QA administration produced a substantial atrophy of the striatum, producing elliptical lesions that encompassed much of the striatum at the level of the injection (FIG. 13A-FIG. 13Q). The size of the lesion was reduced by 86% (t=74.16, df=16, p<0.0001) in animals receiving CP implants (6.18±0.07 versus 0.84±0.02 mm³).

Within the intact contralateral striatum, a general pattern of labeled perikarya was observed that was consistent with previous reports (Emerich et al., J. Neurosci., 16:5168-5181, 1996). The QA lesion significantly reduced the number of ChAT- and diaphorase-positive neurons within the striatum (p's<0.001). CP transplants significantly protected ChAT-positive neurons as only a modest 17% loss of cells was observed relative to controls that showed a 37% loss. Even proximal to the needle tract, many ChAT-positive cells were seen with the typical morphological profile of healthy cholinergic striatal interneurons.

### DISCUSSION

These experiments demonstrate that factors secreted by neonatal CP cells prevent the anatomical and behavioural sequelae seen in an animal model of HD.

### CONCLUSION

The results of the present studies suggest that transplanted choroids plexus cells may be useful in the prevention and/or treatment of a range of acute and chronic CNS diseases.

### Example 8

### Rodent Model of Parkinson's

This example describes the use of neonatal porcine choroid plexus (CP) cells implanted into the brain of rats which have undergone treatment to induce Parkinson's disease to assess the effect of CP implant on neurological function.

### Preparation of Neonatal Porcine CP

Choroid plexus was obtained from newborn (6-15 day) piglets (Auckland Island Strain) and primary cultures of neuroepithelial cells maintained in vitro for 1-3 weeks as described previously (Borlongan C V, Skinner S J, Geaney M, et al., “CNS grafts of rats choroid plexus protect against cerebral ischemia in adult rats,” Neuroreport, 15:1543-7, 2004). The cells were encapsulated in alginate—polyornithine as for Example 7, above, to produce capsules of 600-660 μm diameter. Empty capsules were processed identically and encapsulated cells were maintained in culture for up to 3 weeks prior to transplantation.

### Induction of Parkinsons Disease

Male Sprague-Dawley rats, approximately 9-13 weeks of age and weighing 300-400 grams served as subjects. Animals were housed in a temperature (22±1° C.) and humidity (50±5%) controlled environment and had free access to food and water throughout the study. Rats were anesthetized using isofluorane in oxygen. The striatum was then injected on one side with 6-hydroxydopamine (6-OHDA) using a stereotaxic apparatus for precise co-ordinates. This procedure causes loss of tyrosine hydroxylase-producing cells, resulting in malfunctioning of the substantia nigra and related parts of the central nervous system. The behavioral effect is a relative mild weakness of the contralateral hind and forelimbs when challenged with amphetamine.

At 14 and 28 days after the lesion was initiated, the rats were injected intraperitoneally with amphetamine (5 mg/kg). This drug causes a rapid increase in dopamine production on the un-lesioned side of the brain. The rat becomes hyperactive for 2-3 hours and, if the lesion is of sufficient severity, the rat begins a circling behavioral activity. This behavior was accurately measured using a Rotometer System (San Diego Instruments, San Diego, Calif., USA).

Transplantation of Neonatal Porcine Choroid Plexus Cells and Re-Assessment of Subject Behavior

Rats with persistent lesions (more than 200 circlings/60 min 28 days after administration of 6-OHDA) were separated into control and treatment groups to receive either control implants, consisting of 10 empty capsules, or CP cell implants, consisting of 10 capsules containing choroid plexus cells.

Two days after assignment into control and treatment groups, the capsules were surgically transplanted into the striatum of the rat brains as described in Example 7 using a stereotaxic apparatus (Stoelting Instruments, IL; USA) for precice coordinates.

The rats were again challenged with intraperitoneal injection of amphetamine (5 mg/kg) at 14 and 28 days after transplant surgery. Improvement or deterioration in circling behavior is expressed as % of the mean circling before and after transplant surgery.

Histology

Two to four days after completing the behavioral assessments at 28 days after transplant surgery, the rats were euthanized by CO₂ gas and the brain removed intact and fixed in freshly-prepared buffered paraformaldehyde solution. Thin sections (2 μm) through the striatum were cut and stained for the enzyme tyrosine hydroxylase, essential for dopamine synthesis, using a specific antiserum coupled to a quantitative chemical colorimetric process (Borlongan C V, Skinner S J, Geaney M, et al., “CNS grafts of rats choroid plexus protect against cerebral ischemia in adult rats. (2004) Neuroreport 15:1543-7).

Results

General Observations

No overt signs of behavioral or neurological toxicity were observed in any animals after implantation of either empty or CP-loaded capsules. There were also no adverse effects noted during the postoperative recovery period after 6-OHDA injections, or during the following 30 days period of disease progression. The only noticeable effect was in response to amphetamine injection, as described above. No additional signs of systemic toxicity were noted.

Behavioral Testing

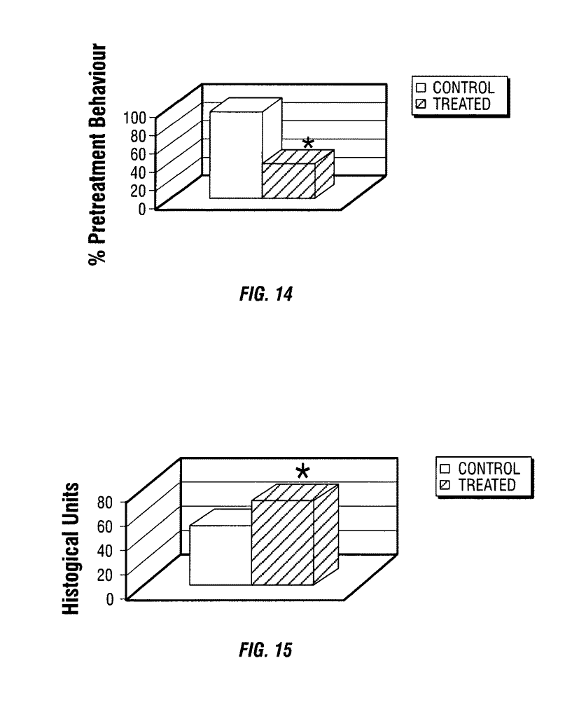

Rats treated with intra-striatal encapsulated CP cells had a 55% decrease in Parkinsonian behavior compared to control rats treated with empty capsules (FIG. 14). The amphetamine-stimulated mean circling behavior decreased by only 5% in the control group whereas the Parkinsonian behavior in the encapsulated CP cell treated group was significantly decreased by 60% (p<0.02).

Histology

Rats treated with intra-striatal encapsulated CP cells had a 33% improvement in tyrosine hydroxylase activity compared to rats in the control group treated with empty capsules (FIG. 15, p<0.05). This increase in tyrosine hydroxylase would lead to greater production of dopamine in the striatum and is expected to contribute to improved muscle control, as observed in the improved behavior (FIG. 14).

These experiments are the first demonstration that treatment with encapsulated porcine choroid plexus can significantly influence recovery of both behavior and neuronal activity in a recognized pre-clinical model of Parkinson's Disease. The 6-OHDA unilateral striatal lesion was allowed to develop for 30 days before either empty capsules (n=9, control) or encapsulated CP cells (n=12, treatment) were transplanted into the rat brains (i.e., after the lesion was established to produce a chronic disease state). The improvement in the CP cell treated rats was substantial. This study demonstrates that encapsulated CP cells can improve recovery of functional neural activity after lesions have been well established for 4-8 weeks, i.e., in a model of chronic disease.

The results of the present studies suggest that transplanted choroids plexus cells may be useful in the prevention and/or treatment of a range of acute and chronic CNS diseases.

### Commercial Benefits or Advantages

The pharmaceutical composition of this invention, when administered to patients suffering from a neurological disease or a disease causing degeneration of the CNS or parts thereof, may slow down or halt the disease (as a palliative treatment). This represents considerable personal, social and economic benefits. We expect that use of choroid plexus cells may even reverse the disease process by providing restorative treatment or possibly stimulating new growth of neurones and/or their processes.

In some instances the invention may simply provide extra CSF; presumably indirectly. In others, it may provide factors that are no longer naturally present in sufficient quantity to maintain neurones against “factors causing atrophy” and in some cases, these factors may be provided only by choroid plexus cells of fetal or neonatal origin.

It may be that future applications simply comprise a rejuvenation of a relatively aged brain; in which instances the use of “new choroid plexus” reverses (to some extent) subclinical ageing processes.

Injuries to the central nervous system may benefit from trophic factors (and possibly also carriers) that can be produced by cell preparations such as those comprising this invention, inserted into the CNS.

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and methods and in the steps or in the sequence of steps of the method described herein without departing from the invention. More specifically, it will be apparent that certain agents, which are both chemically- and physiologically-related may be substituted for the agents described herein while the same or similar results would be achieved. All such similar substitutes and modifications apparent to those skilled in the art are deemed to be within the invention as defined by the appended claims.

1-14. (canceled)

15. An implant comprising living cells collected from the choroid plexus of a porcine, and encapsulated within a biocompatible capsule, at least some of which cells are choroid epithelial cells, the implant secreting at least one product that has a beneficial effect when transplanted into the brain of a recipient mammal suffering from stroke.

16. The implant of claim 15, wherein the biocompatible capsule has at least one inner layer that is effective to serve as a physical substrate for the living cells.

17. The implant of claim 16, wherein the at least one inner layer comprises a laminin.

18. The implant of claim 15, wherein the living cells are comprised within a globular containment that provides orientation and support for the cells.

19. The implant of claim 15, wherein the living cells are comprised within a tubular containment that provides orientation and support for the cells.

20. The implant of claim 15, wherein the at least one product is secreted into at least one region of the brain.

21. The implant of claim 15, wherein the living cells are obtained from the choroid plexus of a fetal or neonatal porcine.

22. The implant of claim 15, wherein at least some of the living cells have undergone a modification to increase the production of the at least one product that has a beneficial effect when transplanted into the brain of a recipient mammal suffering from stroke.

23. The implant of claim 15, wherein the biocompatible capsule comprises alginate.

24. The implant of claim 15, wherein the wall of the capsule is at least partially composed of a semi-permeable membrane that admits metabolites for sustaining the cells, and when implanted in the brain of a recipient mammal suffering from stroke allows an effective amount of one or more expressed products to exit from the implant.

25. The implant of claim 15, wherein the recipient mammal is human.

26. An implant comprising living neonatal- or fetal-derived porcine choroid epithelial cells encapsulated within a biocompatible capsule, the wall of which capsule is at least partially composed of a semi-permeable membrane that admits metabolites for sustaining the cells, and that when implanted within the brain of a recipient mammal suffering from stroke allows an effective amount of one or more expressed products to exit from the implant and access at least one region of the brain to produce a beneficial effect on the mammal.

27. The implant of claim 26, wherein the biocompatible capsule comprises alginate.

28. An anti-stroke implant comprising biocompatible capsules encapsulating living porcine choroid epithelial cells, which when implanted into the brain of a recipient mammal suffering from stroke, is configured to secrete an effective amount of at least one anti-stroke product adapted to prevent, treat, or manage stroke in the mammal.

29. A method of treating, or managing one or more symptoms of stroke in a mammal in need thereof, comprising implanting into the brain of the mammal an implant comprising biocompatible capsules that encapsulate living porcine choroid epithelial cells, which are adapted and configured to secrete an amount of at least one anti-stroke compound effective to treat, or to manage the one or more symptoms of stroke in the mammal.

30. The method of claim 29, wherein the encapsulated living porcine cells are obtained from the choroid plexus of a fetal or a neonatal porcine.

31. A method of preventing stroke in a mammal, comprising: implanting into the brain of the mammal an implant comprising biocompatible capsules that encapsulate living porcine choroid epithelial cells, which are adapted and configured to secrete at least one anti-stroke compound in an amount and time effective to prevent stroke in the mammal.
